(19) United States
(12) Patent Application Publication
(10) Pub. No.: US 2024/0315748 A1
(43) Pub. Date: Sep. 26, 2024

(54) COMPLIANT MECHANISMS FOR ORTHOPAEDIC JOINT REPLACEMENT AND IMPLANTED PROSTHESES

(71) Applicant: THE REGENTS OF THE UNIVERSITY OF CALIFORNIA, Oakland, CA (US)

(72) Inventors: Tyler R. Clites, Calabasas, CA (US); Nelson SooHoo, Los Angeles, CA (US); Jonathan B. Hopkins, Los Angeles, CA (US); Brandon Peterson, Los Angeles, CA (US); Alexander Upfill-Brown, Los Angeles, CA (US)

(21) Appl. No.: 18/737,685

(22) Filed: Jun. 7, 2024

Related U.S. Application Data
(63) Continuation-in-part of application No. PCT/US22/81221, filed on Dec. 8, 2022.
(60) Provisional application No. 63/287,375, filed on Dec. 8, 2021.

(51) Int. Cl. A61B 17/88; A61B 17/56

(52) U.S. Cl. CPC A61B 17/8866; A61B 2017/564

(57) ABSTRACT

The disclosed invention relates to an orthopedic device having a first joint member having an inner surface and an outer surface, a second joint member having an inner surface and an outer surface, and at least one flexure element having first and second ends, wherein the first end is connected to the inner surface of the first joint member and the second end is connected to the inner surface of the second joint member, wherein the first and second joint members are configured to move relative to each other within a first degree of freedom.

### CROSS-REFERENCE TO REPLATED APPLICATIONS

This application is a Continuation-in-Part (CIP) claiming priority to International Application No. PCT/US2022/081221, filed Dec. 8, 2022, which claims priority to U.S. Provisional Patent Application No. 63/287,375 filed Dec. 8, 2021, each of which are incorporated herein by reference in their entirety.

### BACKGROUND OF THE INVENTION

Conventional orthopaedic joint replacements constitute a resurfacing of the damaged joint. As conventional implants are made up of distinct artificial joint surfaces that articulate across each other as the joint moves, joint stability depends on the bone structure and native ligaments surrounding the reconstruction, which limits the amount of deformity that can be corrected. Articulating surfaces also produce synthetic particulates that cause osteolytic response and aseptic loosening, which is the primary failure mode for all joint replacements. Rotating-hinge prostheses produce particulates as well, due to their reliance on articulating loaded surfaces, and are therefore also subject to eventual failure via aseptic loosening. In addition, rotating hinge prostheses are over-constrained, meaning that they are too rigid in all directions other than the single rotating DOF. Conventional joint replacements also depend on robust integration with the native bones surrounding the implant, which is often compromised as a result of the underlying malady, leading to subsidence and implant failure.

The compliance of biological tissue plays an essential role in regulating human movement. Mechanical compliance dictates the body's response to external disturbance, accommodates imperfection, attenuates shock, and allows for storage and return of mechanical energy. This compliance is especially important in joints, which govern how bones move relative to each other. Disruptions of innate joint compliance can dramatically alter movement biomechanics, and have a devastating impact on limb function and quality of life. In some cases, these disruptions occur as a result of injury or disease. Rheumatoid arthritis, for instance, makes joints overly stiff, causing intense pain and increasing the muscular effort required to move. Conversely, some connective tissue disorders, such as Ehlers-Danlos syndrome, cause tissues to become overly soft, which destabilizes joints and leads to recurrent dislocation. In addition to disease or injury, disruptions to biological compliance often occur as a byproduct of the reconstructive treatments sometimes used to correct more severe bone and joint pathologies (FIG. 1A, FIG. 1B, FIG. 1C). For example, in existing systems, the titanium structures used in total joint replacement (FIG. 1A) and constrained endoprostheses (FIG. 1B) are much more rigid than the spongy cancellous bone they replace. This lack of compliance reduces the joint's ability to accommodate biomechanical imperfection which can lead to particulate-driven osteolysis (bone resorption) and aseptic loosening (FIG. 2). In extreme cases, disruptions to biological compliance are necessary for lack of better options. For instance, arthrodesis (joint fusion) is often pursued as a last-resort salvage procedure in cases where state-of-the-art implants are insufficient or have already failed. Arthrodesis involves mechanical fixation of bones such that they fuse together (FIG. 1C), eliminating virtually all compliance in the affected joints. Patients with extensive fusions in their legs face severe limitations in even basic ambulation, cannot run or participate in athletic activities, and are more likely to develop arthritis in their adjacent non-fused joints. These reconstructive procedures are intended to salvage the extremity; however, they can leave patients with a limb that is alive and attached to the body (viable) but unable to support even baseline mobility (nonfunctional). Patients with fusion of the upper extremity face severe limitations in functional tasks which require positioning the arm or grasping with the hand.

Thus, there is a need in the art for a treatment option that would restore joint function, enable a high level of activity, and prevent unnecessary limb loss. The present invention meets this need.

### SUMMARY OF THE INVENTION

In some aspects, the present invention relates to an orthopedic device having a first joint member having an inner surface and an outer surface, a second joint member having an inner surface and an outer surface, and at least one flexure element having first and second ends, wherein the first end is connected to the inner surface of the first joint member and the second end is connected to the inner surface of the second joint member, wherein the first and second joint members are configured to move relative to each other within a first degree of freedom.

In some embodiments, the at least one flexure element has at least a first flexure element and a second flexure element. In some embodiments, the at least one flexure element has at least a first flexure element, a second flexure element and a third flexure element. In some embodiments, the first and third flexure elements are oriented at the same angle relative to each other between the inner surfaces of the first and second joint members. In some embodiments, the second flexure element is positioned between the first and third flexure elements, and wherein the second flexure element is oriented at a different angle relative to the first and third flexure elements between the inner surfaces of the first and second joint members.

In some embodiments, the first joint member has a top region, a bottom region and a central region between the top and bottom region along its length, the second joint member has a top region, a bottom region and a central region between the top and bottom region along its length, the first flexure element is connected at its first end to the inner surface of the bottom region of the first joint member and is connected at its second end to the inner surface of the top region of the second joint member, the second flexure element is connected at its first end to the inner surface of the central region of the first joint member and is connected at its second end to the inner surface of the central region of the second joint member, and the third flexure element is connected at its first end to the inner surface of the bottom region of the first joint member and is connected at its second end to the inner surface of the top region of the second joint member.

In some embodiments, the device further has a first tissue anchor extending from the outer surface of the first joint member, and a second tissue anchor extending from the outer surface of the second joint member. In some embodiments, the first and second tissue anchors are each configured to engage bone via at least one selected from the group consisting of intramedullary fixation, extramedullary fixation, osseointegration and combinations thereof.

In some embodiments, the first and second joint members each have a curvature along its length that includes the top, central and bottom regions. In some embodiments, the first and second joint members are oriented relative to each other such that the top region of the second joint member overlaps the bottom region of the first joint member. In some embodiments, the at least one flexure element is configured to deform when the first joint member moves relative to the second joint member within the first degree of freedom. In some embodiments, the first degree of freedom is translational.

In some embodiments, the at least one flexure element has a plurality of flexure elements. In some embodiments, the plurality of flexure elements are oriented at the same angle relative to each other between the inner surfaces of the first and second joint members. In some embodiments, the plurality of flexure elements are positioned radially and equidistantly about a central axis passing through the first and second joint members.

In some embodiments, the device further has an opening in the first joint member such that the first joint member forms a ring, and a central post having a first end fixed to or contiguous with the inner surface of the second joint member, and a second end extending through the opening of the first joint member such that the first joint member is rotatable around the post. In some embodiments, the device further has a housing surrounding at least the first joint member, the second joint member and the plurality of flexure elements, wherein the first joint member is fixed to the housing.

In some embodiments, the device further has a first tissue anchor connected to the second end of the central post, and a second tissue anchor connected to the outer surface of the second joint member. In some embodiments, the device further has a first tissue anchor connected to the second end of the central post, and a second tissue anchor connected to the housing. In some embodiments, the housing further has at least one recess, and the second joint member further has at least one tab extending from the outer surface of the second joint member and positioned within the at least one recess of the housing, wherein the at least one recess is sized to permit range-limiting movement of the at least one tab in the at least one recess when the second joint member is rotated relative to the first joint member.

In some embodiments, the plurality of flexure elements are configured to deform when the first joint member moves relative to the second joint member within the first degree of freedom. In some embodiments, the first degree of freedom is rotational. In some embodiments, the device further has at least one range-limiting element configured to restrict movement of the first joint member relative to the second joint member within the first degree of freedom. In some embodiments, the device further has at least one range-limiting element configured to restrict movement of the second joint member relative to the first joint member within the first degree of freedom.

In some embodiments, the device further has at least one spring connected to the first and second joint members, wherein the spring is configured to deform when the first and second joint members move relative to each other. In some embodiments, the at least one spring has a first spring and a second spring, wherein the first spring is configured to provide restorative torque to the orthopedic device and the second spring is configured to store and return energy to the orthopedic device.

In some embodiments, the device further has a third joint member having an inner surface and an outer surface, a fourth joint member having an inner surface and an outer surface, at least one flexure element having first and second ends, wherein the first end is connected to the inner surface of the third joint member and the second end is connected to the inner surface of the fourth joint member, wherein the third and fourth joint members are configured to move relative to each other within a second degree of freedom that is different than the first degree of freedom of movement between the first and second joint members, and wherein the outer surface of the third joint member is connected to the outer surface of the second joint member.

In some embodiments, the first and second degrees of freedom are each translational. In some embodiments, the first degree of freedom is translational and the second degree of freedom is rotational. In some embodiments, the first and second degrees of freedom are each rotational. In some embodiments, the device further has a first tissue anchor connected to the outer surface of the first joint member, and a second tissue anchor connected to the outer surface of the fourth joint member. In some embodiments, the first and second tissue anchors are each configured to engage bone via at least one selected from the group consisting of intramedullary fixation, extramedullary fixation, osseointegration and combinations thereof.

In some embodiments, the device further has a third joint member having an inner surface and an outer surface, a fourth joint member having an inner surface and an outer surface, a fifth joint member having an inner surface and an outer surface, a sixth joint member having an inner surface and an outer surface, at least one flexure element having first and second ends, wherein the first end is connected to the inner surface of the third joint member and the second end is connected to the inner surface of the fourth joint member, at least one flexure element having first and second ends, wherein the first end is connected to the inner surface of the fifth joint member and the second end is connected to the inner surface of the sixth joint member, wherein the inner surface of the second joint member is connected to the outer surface of the third joint member, wherein the outer surface of the fourth joint member is connected to the inner surface of the fifth joint member, wherein the third and fourth joint members are configured to move relative to each other within a second degree of freedom that is different than the first degree of freedom of movement between the first and second joint members, and wherein the fifth and sixth joint members are configured to move relative to each other within a third degree of freedom that is different than the second degree of freedom of movement between the third and fourth joint members.

In some embodiments, the first degree of freedom is translational, the second degree of freedom is rotational, and the third degree of freedom is translational. In some embodiments, the first degree of freedom is rotational, the second degree of freedom is translational, and the third degree of freedom is rotational. In some embodiments, the first, second and third degrees of freedom are each translational. In some embodiments, the first, second and third degrees of freedom are each rotational. In some embodiments, the device further has a first tissue anchor extending from the outer surface of the first joint member, and a second tissue anchor extending from the outer surface of the sixth joint member. In some embodiments, the first and second tissue anchors are each configured to engage bone via at least one selected from the group consisting of intramedullary fixation, extramedullary fixation, osscointegration and combinations thereof.

In some aspects, the present invention relates to a method of designing compliant implantable prostheses having the steps of performing neuromusculoskeletal (NMS) modeling on a subject to identify anatomical constraints and desired degree of freedom compliance for an implant, generating a rough approximation of a mechanism geometry required to produce the desired compliance space by using freedom and constraint topologies (FACT), creating a parameterized finite element model (FEM) of the generic mechanism; from a quasi-static mechanic's model of the implant under loads representative of those encountered during gait, and refining mechanism geometry by adjusting shape of the implant.

### BRIEF DESCRIPTION OF THE DRAWINGS

The following detailed description of embodiments of the invention will be better understood when read in conjunction with the appended drawings. It should be understood, however, that the invention is not limited to the precise arrangements and instrumentalities of the embodiments shown in the drawings.

FIG. 1A through FIG. 1C depicts current treatments for limb pathology. FIG. 1A depicts joint replacement which relies on ligaments for stability and is prone to rubbing/sliding wear. FIG. 1B depicts constrained endoprostheses which is inherently stable and is prone to rubbing/sliding wear and over-constraint. FIG. 1C depicts fusion therapy which is stable and viable but nonfunctional. All these treatment options produce unnatural limb compliance.

FIG. 2 depicts a rotating hinge total knee prosthesis.

FIG. 3A through FIG. 3B depicts a 1-degree of freedom implementation of an exemplary implant device of the present invention. FIG. 3A depicts an undeformed exemplary implant device of the present invention. FIG. 3B depicts a deformed exemplary implant device of the present invention.

FIG. 3C through FIG. 3L depicts various embodiments of compliant mechanisms for an implant device of the present invention. FIG. 3C depicts a side view of a compliant mechanism for an implant device of the present invention. FIG. 3D depicts a perspective view of a compliant mechanism for an implant device of the present invention. FIG. 3E depicts a side view of an exemplary 3-piece compliant mechanism for an implant device of the present invention. FIG. 3F depicts a perspective view of an exemplary 3-piece compliant mechanism for an implant device of the present invention. FIG. 3G depicts an exploded perspective view of an exemplary 3-piece compliant mechanism for an implant device of the present invention. FIG. 3H depicts a finite element analysis for ankle loading validation of an exemplary undeformed compliant mechanism for an implant device of the present invention. FIG. 3I depicts a finite element analysis for ankle loading validation of an exemplary deformed compliant mechanism for an implant device of the present invention. FIG. 3J depicts a perspective view of a compliant mechanism for an implant device of the present invention. FIG. 3K depicts a side view of a compliant mechanism for an implant device of the present invention. FIG. 3L depicts a finite element analysis for ankle loading validation of an exemplary deformed compliant mechanism for an implant device of the present invention.

FIG. 4A through FIG. 4F depicts an exemplary implant device of the present invention applied to the ankle joint. FIG. 4A depicts a side view of an exemplary undeformed implant device of the present invention applied to the ankle joint. FIG. 4B depicts a perspective view of an exemplary undeformed implant device of the present invention applied to the ankle joint. FIG. 4C depicts a perspective view of an exemplary undeformed implant device of the present invention applied to the ankle joint. FIG. 4D depicts a perspective view of an exemplary undeformed implant device of the present invention with a cover to protect the surrounding tissue of the ankle joint. FIG. 4E depicts a perspective view of an exemplary undeformed implant device of the present invention applied to the ankle joint. FIG. 4F depicts a side view of an exemplary undeformed implant device of the present invention applied to the ankle joint.

FIG. 5A through FIG. 5D depicts an exemplary implant device of the present invention. FIG. 5A depicts a perspective view of an exemplary undeformed implant device of the present invention. FIG. 5B depicts a perspective view of an exemplary undeformed implant device of the present invention. FIG. 5C depicts a side view of an exemplary undeformed implant device of the present invention. FIG. 5D depicts a finite element analysis of ISO standard ankle loads of an exemplary implant device of the present invention.

FIG. 6A through FIG. 6B depicts an exemplary modular implant device of the present invention. FIG. 6A depicts an exploded perspective view of an exemplary modular implant device of the present invention applied to the ankle joint. FIG. 6B depicts a perspective view of an assembled exemplary modular implant device of the present invention applied to the ankle joint.

FIG. 7 depicts a finite element analysis of ISO standard ankle loads of an exemplary implant device of the present invention.

FIG. 8A through FIG. 8B depicts an exemplary implant device of the present invention. FIG. 8A depicts an exemplary 2-degree of freedom implant device of the present invention applied to the ankle and subtalar joint. FIG. 8B depicts an exemplary 2-degree of freedom implant device of the present invention applied to the ankle and subtalar joint that is deformed under load.

FIG. 9 depicts perspective views of an exemplary 2-degree of freedom implant device of the present invention.

FIG. 10A through FIG. 10D depicts an exemplary 2-degree of freedom implant device of the present invention applied to the ankle and subtalar joint. FIG. 10A depicts a lateral view of an exemplary undeformed device of the present invention. FIG. 10B depicts an anterior view of an exemplary undeformed device of the present invention. FIG. 10C depicts a posterior view of an exemplary undeformed device of the present invention. FIG. 10D depicts a medial view of an exemplary undeformed device of the present invention.

FIG. 11A through FIG. 11D depicts an exemplary 2-degree of freedom implant device of the present invention applied to the ankle and subtalar joint. FIG. 11A depicts a lateral view of an exemplary deformed device of the present invention (dorsiflexion). FIG. 11B depicts an anterior view of an exemplary deformed device of the present invention (eversion). FIG. 11C depicts a posterior view of an exemplary deformed device of the present invention (inversion). FIG. 11D depicts a medial view of an exemplary deformed device of the present invention (plantar flexion).

FIG. 12 depicts an exemplary 1-degree of freedom implant device of the present invention designed for moderate compressive loads and high range of motion.

FIG. 13 depicts an exemplary coil spring that can be used in parallel with a implant device of the present invention to increase energy storage capacity.

FIG. 14A through FIG. 14D depicts exemplary 2 degree of freedom implementations of the implant architectures. FIG. 14A depicts a cross-axis pivot architecture. FIG. 14B depicts a cable suspended architecture. FIG. 14C depicts a parallel leaf spring and a cross axis pivot architecture. FIG. 14D depicts a cross-axis pivot architecture.

FIG. 15A is a perspective view of an exemplary implant device of the present invention. FIG. 15B depicts a finite element analysis of rotational loading of an exemplary implant device of the present invention. FIG. 15C depicts a finite element analysis of rotational loading of an exemplary implant device of the present invention.

FIG. 16 is a perspective view of an exemplary implant device of the present invention.

FIG. 17A depicts exploded perspective and cut away views of an exemplary implant device of the present invention. FIG. 17B depicts cut-away views and a perspective view of an exemplary compliant mechanism for an exemplary implant device of the present invention. FIG. 17C depicts cut away views and a perspective view of an exemplary implant device of the present invention. FIG. 17D depicts cut-away views and a perspective view of an exemplary compliant mechanism for an exemplary implant device of the present invention. FIG. 17E depicts a top down of an exemplary implant device of the present invention.

FIG. 18A depicts exploded side views of an exemplary implant device of the present invention. FIG. 18B depicts a finite element analysis of rotational loading of an exemplary implant device of the present invention.

FIG. 19A through FIG. 19D depicts an exemplary implant device of the present invention. FIG. 19A depicts a side view of an exemplary implant device of the present invention. FIG. 19B depicts a bottom perspective view of an exemplary implant device of the present invention. FIG. 19C depicts a top perspective view of an exemplary implant device of the present invention. FIG. 19D depicts a bottom perspective view of an exemplary implant device of the present invention.

FIG. 19E through FIG. 19H depicts an exemplary implant device of the present invention. FIG. 19E depicts a perspective view of an exemplary implant device of the present invention. FIG. 19F depicts a perspective view of a cross-section of an exemplary implant device of the present invention. FIG. 19G depicts a side view of a cross-section of an exemplary implant device of the present invention. FIG. 19H depicts a perspective cutaway view of an exemplary implant device of the present invention without an outer shell.

FIG. 20A through FIG. 20H depicts an exemplary 2-degree of freedom implant device of the present invention. FIG. 20A depicts a side view of an exemplary implant device of the present invention. FIG. 20B depicts a perspective view of an exemplary implant device of the present invention. FIG. 20C depicts a lateral view of an exemplary implant device of the present invention. FIG. 20D depicts an anterior view of an exemplary implant device of the present invention. FIG. 20E depicts a perspective view of an exemplary implant device of the present invention. FIG. 20F depicts a lateral view of an exemplary implant device of the present invention. FIG. 20G depicts a perspective view of an exemplary implant device of the present invention. FIG. 20H depicts a posterior view of an exemplary implant device of the present invention.

FIG. 21A through FIG. 21D depict finite element analysis of loading of an exemplary implant device of the present invention. FIG. 21A depicts finite element analysis of loading of an exemplary undeformed implant device of the present invention. FIG. 21B depicts finite element analysis of loading of an exemplary implant device of the present invention. FIG. 21C depicts finite element analysis of loading of an exemplary deformed implant device of the present invention. FIG. 21D depicts finite element analysis of loading of an exemplary deformed implant device of the present invention.

FIG. 22A through FIG. 22G depicts an exemplary 3-degree of freedom implant device of the present invention. FIG. 22A depicts a perspective view of an exemplary 3-degree of freedom implant device of the present invention. FIG. 22B depicts a lateral view of an exemplary 3-degree of freedom implant device of the present invention. FIG. 22C depicts a perspective view of an exemplary 3-degree of freedom implant device of the present invention. FIG. 22D depicts a side cutaway view of an exemplary 3-degree of freedom implant device of the present invention. FIG. 22E depicts a perspective cutaway view of an exemplary 3-degree of freedom implant device of the present invention. FIG. 22F depicts a top-down view of an exemplary 3-degree of freedom implant device of the present invention. FIG. 22G depicts a side view of an exemplary 3-degree of freedom implant device of the present invention.

FIG. 23A through FIG. 23L depicts an exemplary implant device of the present invention for an animal model. FIG. 23A depicts an exploded perspective view of an exemplary implant device of the present invention for an animal model. FIG. 23B depicts a side view of an exemplary implant device of the present invention for an animal model. FIG. 23C depicts a side view of an exemplary implant device of the present invention for an animal model with a cover to protect the surrounding tissue of the joint. FIG. 23D depicts a side view of an exemplary implant device of the present invention for an animal model without a cover. FIG. 23E depicts an enlarged side view of an exemplary implant device of the present invention for an animal model with a cover to protect the surrounding tissue of the joint. FIG. 23F depicts an enlarged side view of an exemplary implant device of the present invention for an animal model without a cover. FIG. 23G depicts a perspective view of an exemplary implant device of the present invention for an animal model without a cover. FIG. 23H depicts a perspective view of an exemplary implant device of the present invention for an animal model with a cover to protect the surrounding tissue of the joint. FIG. 23I depicts an enlarged perspective view of an exemplary implant device of the present invention for an animal model without a cover. FIG. 23J depicts an enlarged perspective view of an exemplary implant device of the present invention for an animal model with a cover to protect the surrounding tissue of the joint. FIG. 23K depicts an enlarged perspective view of an exemplary implant device of the present invention for an animal model. FIG. 23L depicts an enlarged side view of an exemplary implant device of the present invention for an animal model.

FIG. 24 depicts a schematic of an exemplary device of the present invention in use with an external exoskeleton.

FIG. 25 is a flowchart depicting an exemplary method for facilitating the design of compliant implants for limb restoration.

FIG. 26A through FIG. 26B depicts exemplary drill guides for the implantation of at least one anchor or rod of the present invention. Shown in FIG. 26A is a calcaneal drill guide. Shown in FIG. 26B is a midfoot drill guide.

FIG. 27A through FIG. 27F depicts preliminary modeling and experimental work of an exemplary implant device of the present invention. FIG. 27A depicts an ankle-hindfoot implant design. FIG. 27B depicts a finite element analysis of ISO standard ankle loads. FIG. 27C depicts benchtop evaluation of preliminary mechanisms. FIG. 27D depicts benchtop evaluation of preliminary mechanisms. FIG. 27E depicts benchtop evaluation of preliminary mechanisms. FIG. 27F depicts cadaveric dissection to assess potential distal fixation site.

FIG. 28 depicts cadaveric dissection and placement of an exemplary implant device of the present invention.

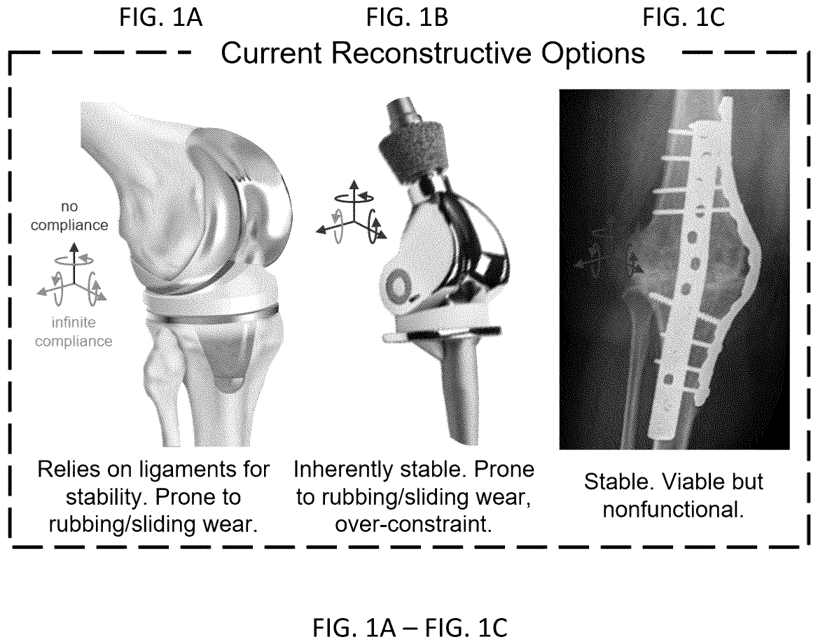

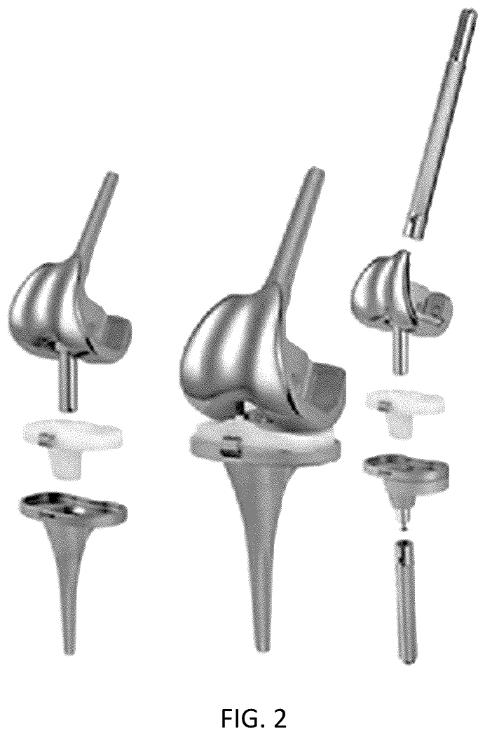

### DETAILED DESCRIPTION

It is to be understood that the figures and descriptions of the present invention have been simplified to illustrate elements that are relevant for a clear understanding of the present invention, while eliminating, for the purpose of clarity many other elements found in the field of orthopedic joint replacement and implanted prostheses. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, exemplary materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate. The terms “patient,” “subject,” “individual,” and the like are used interchangeably herein, and refer to any animal amenable to the systems, devices, and methods described herein. The patient, subject or individual may be a mammal, and in some instances, a human.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The term “connected to” as used herein may mean connected to by direct contact, or connected to mechanically via one or more intermediate components.

### Implant for Orthopedic Joint Replacement and Implanted Prosthesis

The present invention provides in part orthopedic implants comprising one or more compliant mechanisms. As contemplated herein, a compliant mechanism utilizes a flexible structure, also described herein as a flexure element, that transmits motion through flexing and/or elastic body deformation of the flexible structure. In some embodiments, compliant mechanisms achieve their motion from the relative flexibility of the structure's members rather than from rigid-body joints alone. In some embodiments, compliant mechanisms can be designed to permit motion in some directions (both linear and rotational), and to be functionally rigid in other directions. In some embodiments, compliant mechanisms can be composed of a single piece, or of multiple pieces connected together. For example, a compliant mechanism may include at least one flexible structure. However, it should be appreciated that a compliant mechanism may include any number of flexible structures. For example, a compliant mechanism may include at least two flexible structures, at least three flexible structures, at least four flexible structures, at least five flexible structures, at least six flexible structures, at least seven flexible structures, at least eight flexible structures, at least nine flexible structures, at least ten flexible structures, at least twenty flexible structures, at least fifty flexible structures, or at least one hundred flexible structures. In some embodiments, the orthopedic implant devices of the present invention are configured to replace or enhance biological joint function. In some embodiments, the devices of the present invention are configured to allow flexible movement in one or more engineered degrees of freedom (both translational (e.g. linear) and rotational) and rigidity in all remaining degrees of freedom. This configuration may allow for increased longevity and joint stability. In some embodiments, the devices of the present invention are configured to re-engineer limb compliance. In some embodiments, the devices of the present invention are configured to correct pathological compliance. In some embodiments, the devices of the present invention alleviate pain. In some embodiments, the devices of the present invention are configured to prevent amputation. In some embodiments, the devices of the present invention mimic or augment innate biological compliance and thereby restore function to a joint and eliminate pain. In some embodiments, the devices of the present invention consist of flexible elements that deform under load to produce desired motion. In some embodiments, the devices of the present invention precisely guide motion without the mechanical complexity of bearings and linkages. In some embodiments, the devices of the present invention are essentially frictionless or produce negligible friction, and therefore generate negligible wear. In some embodiments, the devices of the present invention have inherent stability, which is important because it allows compromised bones to be resected without destabilizing the joint. In some embodiments, the devices of the present invention store energy and generate restorative torques, opening the door to implanted devices that function as prostheses, restoring function of lost limb segments, or orthoses, enhancing or correcting function in intact biological limbs. This mechanism benefits localized muscle pathology by decreasing the compensatory muscle function necessary to support gait. In some embodiments, the devices of the present invention are configured to withstand cyclic gait loads.

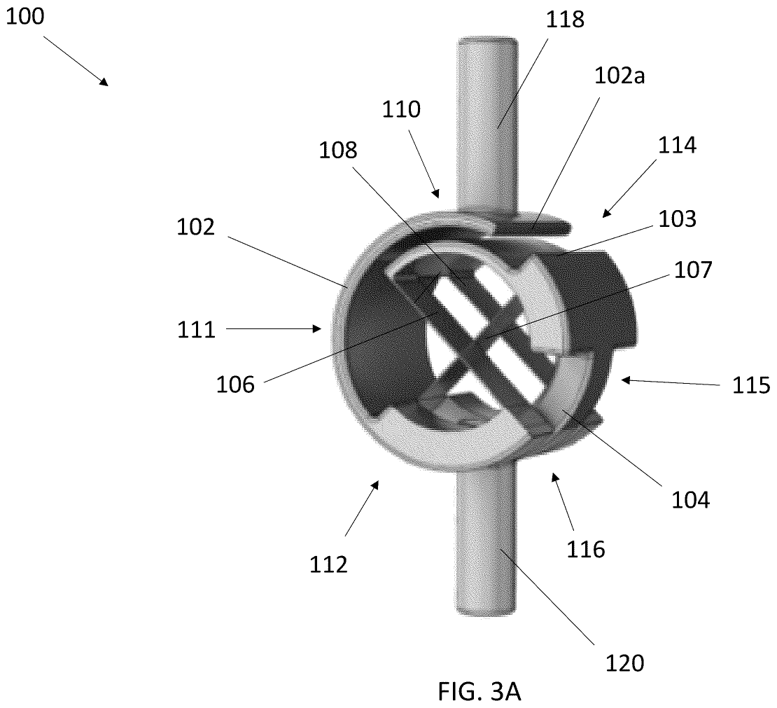

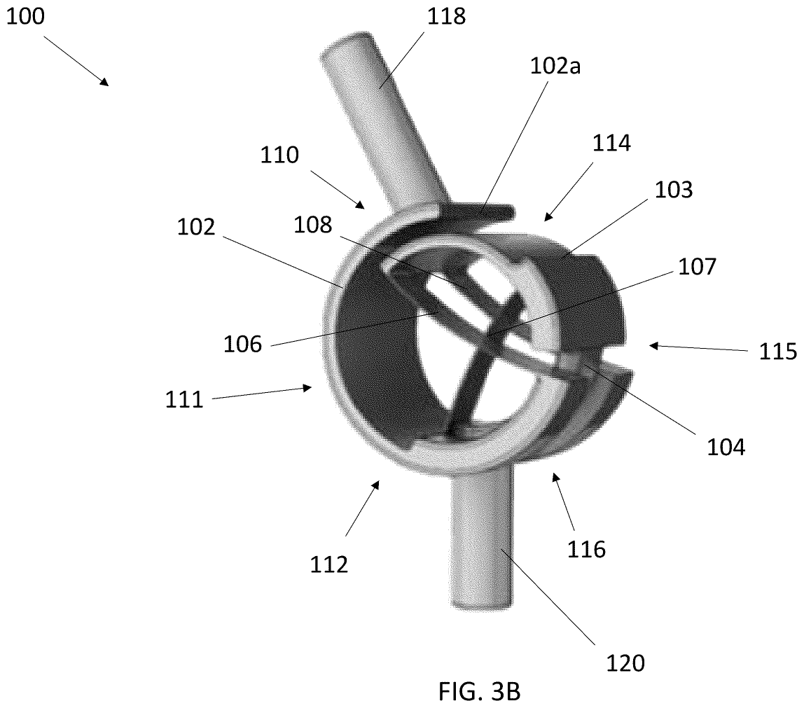

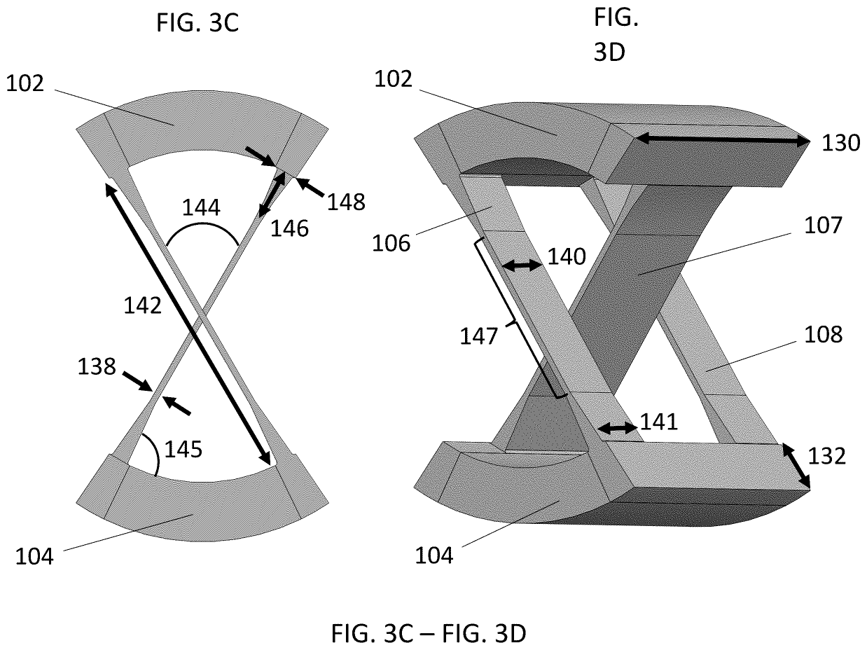

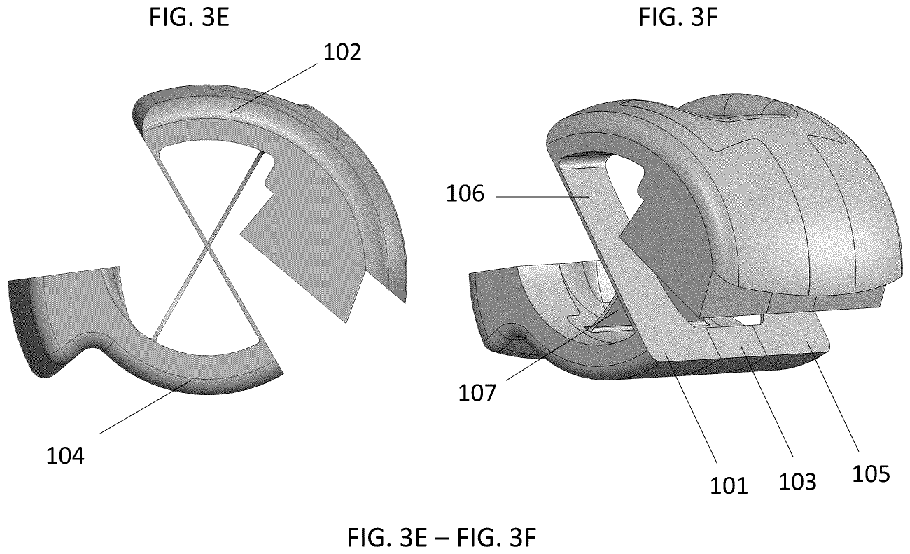

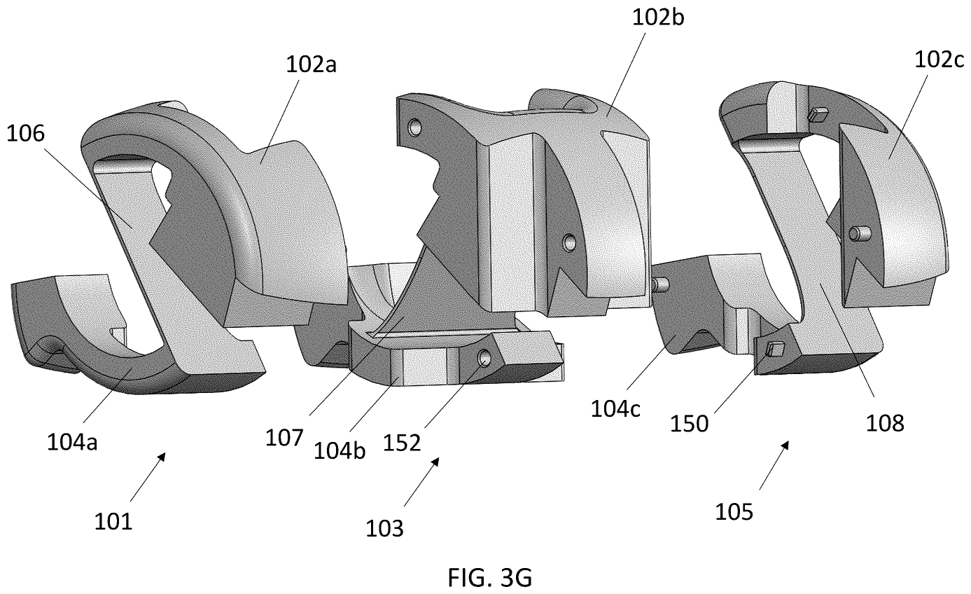

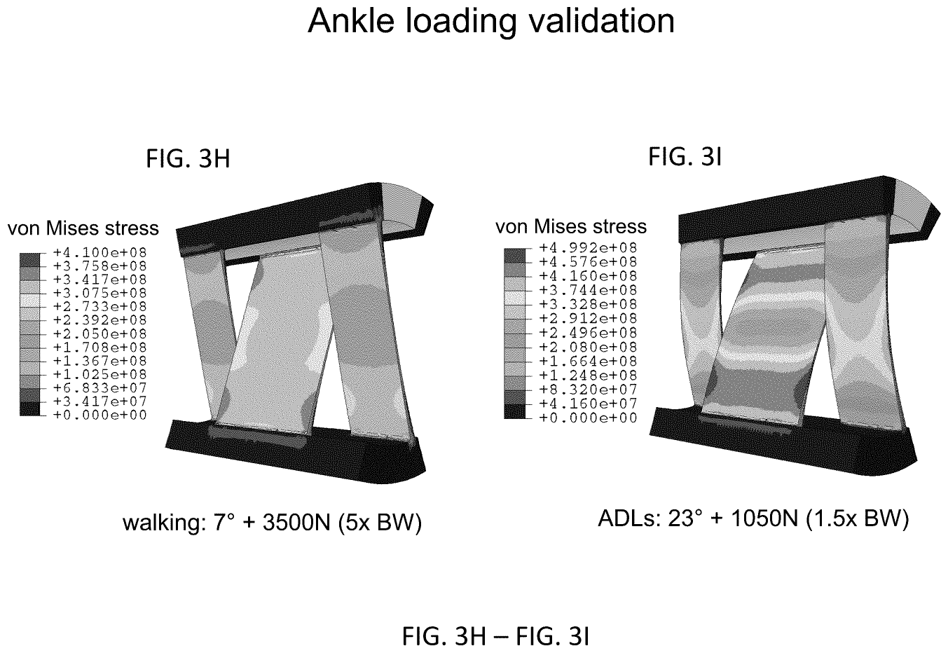

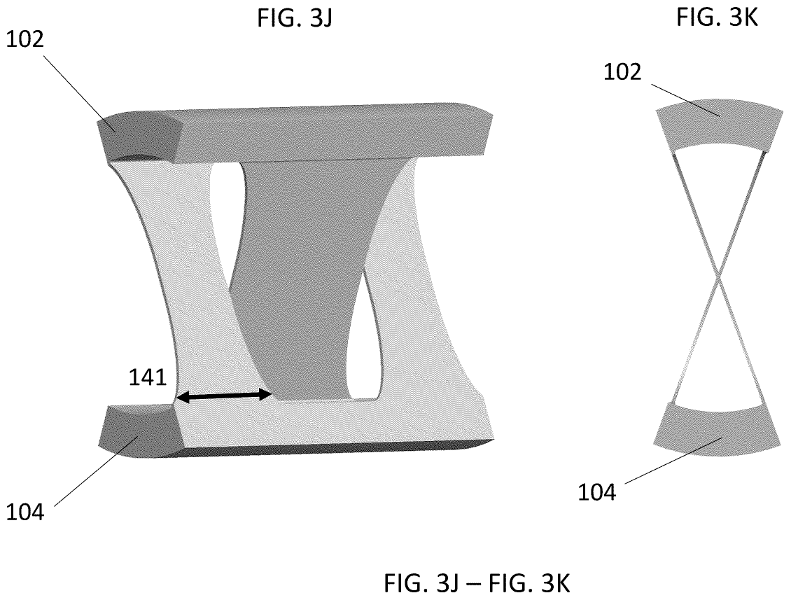

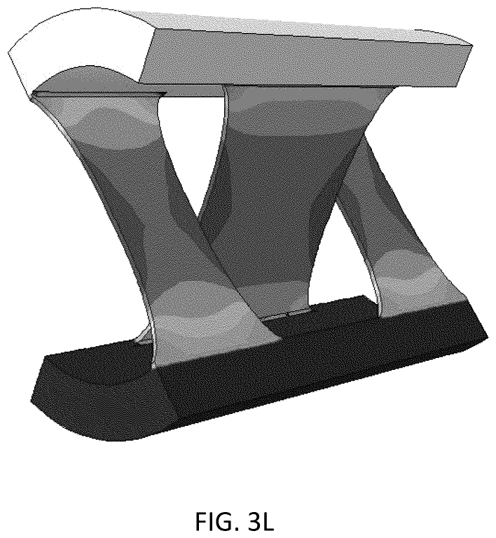

Referring now to FIG. 3A and FIG. 3B, an exemplary implant device having a cross-axis flexural pivot is shown. In some embodiments, device 100 comprises a first joint member 102, a second joint member 104, at least one flexure element (e.g. first flexure element 106, second flexure element 107, and third flexure clement 108), a proximal anchor 118 and a distal anchor 120.

In some aspects, the present invention relates to an orthopedic device 100 comprising a first joint member 102 having an inner surface and an outer surface, a second joint member 104 having an inner surface and an outer surface; and at least one flexure element having first and second ends, wherein the first end is connected to the inner surface of first joint member 102 and the second end is connected to the inner surface of second joint member 104, wherein the first and second joint members are configured to move relative to each other within a first degree of freedom.

In some embodiments, first joint member 102 and second joint member 104 may have any shape known to one skilled in the art. In some embodiments, first joint member 102 and second joint member 104 may have a circular or rounded outer geometry to allow first joint member 102 and second joint member 104 to rotate cleanly about a center of rotation. In some embodiments, first joint member 102 has a larger inner diameter than second joint member 104. In some embodiments, this configuration has hard-stops to limit the motion to a range that would not over-stress at least one flexure element (e.g. first flexure element 106, second flexure element 107, third flexure clement 108). In some embodiments, first joint member 102 and second joint member 104 may have any thickness ranging from approximately 0.05-10 mm. In some embodiments, first joint member 102 and second joint member 104 may have any width or diameter ranging approximately between 0.5-20 cm, depending on the joint. In some embodiments, thickness, width and/or diameter of first joint member 102 and second joint member 104 may be modified to change overall behavior including but not limited to range of motion, load bearing capacity, stiffness, center of rotation trajectory, etc. In some embodiments, first joint member 102 and second joint member 104 have variable thickness, width and/or diameter along their length. In some embodiments, first joint member 102 and second joint member 104 have uniform thickness, width, and/or diameter along their length. In some embodiments, first joint member 102 and/or second joint member 104 are substantially c-shaped to provide a specific stiffness in joint compression.

In some embodiments, device 100 may further comprise at least one range-limiting element 103 configured to restrict motion, such that device 100 is not over-stressed or over-extended. In some embodiments, the at least one range-limiting element 103 comprises a raised or extended portion along the length of second joint member 104 thereby forming a hard stop configured to block further movement of a movable element such as a leading edge 102a of first joint member 102. The hard stop may have any shape known to one skilled in the art. For example, the range-limiting element 103 may present one or more flat surfaces or faces against which a respective flat surface or face of a movable element presses against at the end of a range of motion. In some embodiments, the at least one range-limiting element 103 comprises one or more hooks or overhangs against which a respective hook or overhang of a movable element catches onto at the end of a range of motion. In some embodiments, the at least one range-limiting element 103 comprises one or more linear or non-linear stiffening flexure element, such as a spring or compliant mechanism, wherein the flexure element comprises a range of motion that is restricted at range ends, such as in total or partial compression and/or tension. In other embodiments, the range-limiting elements may include tabs or other extensions that fit within a corresponding opening or recess, such that when the tab or other extension makes contact with the wall of the opening or recess, movement is restricted.

First joint member 102 and second joint member 104 are configured to form a cross-axis pivot flexure, wherein first joint member 102 and second joint member 104 are oriented respectively such that their movement is along the same plane, allowing 1 degree of freedom. First joint member 102 comprises a top region 110, a central region 111, and a bottom region 112. Top region 110 is connected to proximal anchor 118 and is configured to move in the same plane relative to second joint member 104. Second joint member 104 comprises a top region 114, a central region 115, and a bottom region 116. Bottom region 116 is connected to distal anchor 120 and is configured to move in the same plane relative to first joint member 102. Device 100 is configured such that bottom region 116 of second joint member 104 is nested within bottom region 112 of first joint member 102. In some embodiments, top region 110 of first joint member 102 overlays top region 114 of second joint member 104. In some embodiments, first joint member 102 rotates within second joint member 104.

Other hardstops or range-limiting elements are contemplated herein. In some embodiments, second joint member 104 can be designed to press against the sides of bottom region 116 to prevent too much stress on the mechanism from out-of-plane loads (for example a force into the page on distal anchor 120). In some embodiments, a hard stop could be placed between top region 110 and top region 114, such that portions interfere during large compressive loads, and prevent overstressing the flexure elements in tension. Such hardstops may comprise a different material from the rest of the implant, such as ultra-high molecular weight polyethylene.

In some embodiments, first joint member 102 comprises a top region 110, a bottom region 112 and a central region 111 between the top and bottom region along its length, second joint member 104 comprises a top region 114, a bottom region 116 and a central region 115 between the top and bottom region along its length, first flexure element 106 is connected at its first end to the inner surface of the bottom region of first joint member 102 and is connected at its second end to the inner surface of the top region of second joint member 104, second flexure element 107 is connected at its first end to the inner surface of the central region of first joint member 102 and is connected at its second end to the inner surface of the central region of second joint member 104, and third flexure element 108 is connected at its first end to the inner surface of the bottom region of first joint member 102 and is connected at its second end to the inner surface of the top region of second joint member 104.

In some embodiments, first joint member 102 and second joint member 104 each have a curvature along its length that includes the top, central and bottom regions. In some embodiments, first joint member 102 and second joint member 104 are oriented relative to each other such that the top region of second joint member 104 overlaps the bottom region of first joint member 102.

In some embodiments, the at least one flexure element comprises a first end of the flexure element connected to the first joint member and a second end of the flexure element connected to the second joint member. In some embodiments, device 100 may have any number of flexure elements, where each flexure element forms a connection between first and second joint members. In some embodiments, the at least one flexure element comprises a first flexure element 106, a second flexure element 107, and a third flexure element 108, where each flexure element 106, 107 and 108 has a respective first end connected to the first joint member and a respective second end connected to the second joint member. For example, first flexure element 106 and third flexure element 108 connect bottom region 112 of first joint member 102 to top region 114 of second joint member 104. In some embodiments, the connection points of first and third flexure elements 106 and 108 may be at the leading edges of first and second joint members 102 and 104, as shown in FIG. 3E. Second flexure element 107 connects first joint member 102 to second joint member 104 at points along their respective circular arc lengths. First flexure element 106 and third flexure element 108 may have the same structure and angular orientation, with second flexure element 107 being disposed between first flexure element 106 and third flexure element 108 and positioned at an angle different from the first and second flexure elements 106 and 108. In some embodiments, the at least one flexure element is formed as one unit with first joint member 102 and/or second joint member 104. In some embodiments, the at least one flexure element is formed separately from first joint member 102 and/or second joint member 104, and is later connected to first joint member 102 and/or second joint member 104.

In some embodiments, the at least one flexure element is configured to deform when first joint member 102 moves relative to second joint member 104 within the first degree of freedom. In some embodiments, the first degree of freedom is translational. In some embodiments, the first degree of freedom is rotational. In some embodiments, device 100 further comprises a first tissue anchor (e.g. proximal anchor 118) extending from the outer surface of first joint member 102, and a second tissue anchor (e.g. distal anchor 120) extending from the outer surface of second joint member 104. In some embodiments, the first and second tissue anchors are each configured to engage bone via at least one selected from the group consisting of intramedullary fixation, extramedullary fixation, osscointegration and combinations thereof.

FIG. 3C, FIG. 3D, FIG. 3J and FIG. 3K illustrate a close-up of a compliant mechanism similar to that shown in FIGS. 3A and 3B and utilizes the same numbered parts for simplicity of description. In some embodiments, device 100 comprises first joint member 102 and second joint member 104 having a width 130 and a thickness 132. In some embodiments, first joint member 102 and second joint member 104 may have any thickness 132 ranging approximately between 0.05-10 mm. In some embodiments, first joint member 102 and second joint member 104 may have any width 130 or diameter ranging approximately between 0.5-20 cm, depending on the joint. In some embodiments, width, thickness, and diameter of first joint member 102 and second joint member 104 may be modified to change overall behavior including but not limited to range of motion, load bearing capacity, stiffness, center of rotation trajectory, etc., depending on the type of joint being replaced. In some embodiments, first joint member 102 and second joint member 104 have variable width, thickness and/or diameter along their length. In some embodiments, first joint member 102 and second joint member 104 have uniform width, thickness and/or diameter along their length.

As described herein, the one or more flexure elements may also be referred to as blades. In some embodiments, device 100 comprises at least one flexure element or blade, wherein the element or blade has a thickness 138, a width 140, a length 142, a relative angle 144, joint member angle 145, a fillet length 146 and a fillet width 141. In some embodiments, first flexure element 106, second flexure element 107 and third flexure element 108 may have the same thickness. In some embodiments, first flexure element 106 and third flexure element 108 may be thicker compared to second flexure element 107. In some embodiments, second flexure element 107 may be thicker compared to first flexure element 106 and third flexure element 108. In some embodiments, first flexure element 106, second flexure element 107 and third flexure element 108 may have any thickness 138 ranging approximately between 0.005-50 mm. In some embodiments, first flexure element 106, second flexure element 107 and third flexure element 108 may have varying thicknesses along their length. For example, a blade may include a fillet or thicker region 146 along its length, such that the blades have an increased thickness 138 at the connection point to the first or second joint members 102 or 104. In such embodiments, the blade utilizes a thinner central region 147 of its length to promote the desired deformation during flexure. In some embodiments, first flexure element 106, second flexure element 107 and third flexure element 108 may have the same width. In some embodiments, first flexure element 106 and third flexure element 108 may be wider compared to second flexure element 107. In some embodiments, second flexure element 107 may be wider compared to first flexure element 106 and third flexure element 108. In some embodiments, first flexure element 106, second flexure element 107 and third flexure element 108 may have varying widths along their length. In some embodiments, first flexure element 106 and third flexure clement 108 may have any width 140 ranging approximately between 0.1-10 cm. In some embodiments, second flexure element 107 may have any width 140 ranging approximately between 0.1-10 cm. In some embodiments, first flexure element 106, second flexure element 107 and third flexure element 108 may have the same length. In some embodiments, first flexure element 106 and third flexure element 108 may be longer compared to second flexure element 107. In some embodiments, second flexure clement 107 may be longer compared to first flexure element 106 and third flexure element 108. In some embodiments, first flexure element 106 and third flexure element 108 may have any length 142 ranging approximately between 0.2-20 cm. In some embodiments, second flexure element 107 may have any length 142 ranging approximately between 0.2-20 cm. In some embodiments, thickness 138, width 140, and length 142 of first flexure element 106, second flexure element 107 and third flexure element 108 may be modified to change overall behavior including but not limited to range of motion, load bearing capacity, stiffness, center of rotation trajectory, etc. In some embodiments, thickness 138, width 140, and length 142 of first flexure element 106, second flexure element 107 and third flexure element 108 may vary in at least one region of the flexure element.

In some embodiments, any two flexure elements may have an orientation of any angle 144 relative to each other. For example, angle 144 may be between 0°-180°, between 0°-90°, between 0°-60°, between 0°-45°, between 0°-30°, between 0°-15°, or between 0°-10°. In some embodiments, angle 144 may less than or equal to 180°, less than or equal to 170°, less than or equal to 160°, less than or equal to 150°, less than or equal to 140°, less than or equal to 130°, less than or equal to 120°, less than or equal to 110°, less than or equal to 100°, less than or equal to 90°, less than or equal to 80°, less than or equal to 70°, less than or equal to 60°, less than or equal to 50°, less than or equal to 40°, less than or equal to 30°, less than or equal to 20°, less than or equal to 10°, less than or equal to 5°, less than or equal to 1°, or about 0°.

In some embodiments, the first and third flexure elements are oriented at the same joint member angle 145 relative to each other between the inner surfaces of of first joint member 102 and second joint member 104. In some embodiments, second flexure element 107 is positioned between the first flexure element 106 and third flexure element 108, and wherein second flexure element 107 is oriented at a different angle relative to first flexure element 106 and third flexure element 108 between the inner surfaces of first joint member 102 and second joint member 104.

In some embodiments, any flexure element may have an orientation of any joint member angle 145 relative to any joint member. For example, angle 145 may be between 0°-180°, between 0°-90°, between 0°-60°, between 0°-45°, between 0°-30°, between 0°-15°, or between 0°-10°. In some embodiments, angle 144 may less than or equal to 180°, less than or equal to 170°, less than or equal to 160°, less than or equal to 150°, less than or equal to 140°, less than or equal to 130°, less than or equal to 120°, less than or equal to 110°, less than or equal to 100°, less than or equal to 90°, less than or equal to 80°, less than or equal to 70°, less than or equal to 60°, less than or equal to 50°, less than or equal to 40°, less than or equal to 30°, less than or equal to 20°, less than or equal to 10°, less than or equal to 5°, less than or equal to 1°, or about 0°.

In some embodiments, the flexure elements may comprise at least one fillet having any fillet length 146 ranging approximately between 0.1-50 mm. In some embodiments, the flexure elements may have any fillet width 141 ranging approximately between 0.1-50 mm. In some embodiments, the flexure elements may have any fillet thickness 138 ranging approximately between 0.1-50 mm. In some embodiments, the flexure elements may have any fillet radius ranging approximately between 0.1 mm-10 cm.

Aspects of the present invention relate to a compliant mechanism with a multi-piece assembly for an implant device of the present invention. FIG. 3E through FIG. 3G illustrate a close-up of a compliant mechanism similar to that shown in FIGS. 3A and 3B and utilizes the same numbered parts for simplicity of description. Shown in FIG. 3E and FIG. 3F is the assembled compliant mechanism. Shown in FIG. 3G is the exploded, disassembled view of the compliant mechanism. In some embodiments, the compliant mechanism comprises at least one major component. For example, in some embodiments, the compliant mechanism comprises a first component 101, second component 103 and a third component 105. In some embodiments, first component 101 comprises a first joint member 102a and a second joint member 104a connected by a first flexure element 106. In some embodiments, second component 103 comprises a first joint member 102b and a second joint member 104b connected by a second flexure element 107. In some embodiments, third component 105 comprises a first joint member 102c and a second joint member 104c connected by a third flexure element 108. In some embodiments, first component 101, second component 103 and third component 105 have mating surfaces allowing for a friction fit of the components when assembled. In some embodiments, the compliant mechanism has features for securing the components together with a friction fit, such as, but not limited to, slots, grooves, holes, channels, notches, tongues, pins, pegs, tangs, keys, blades, and/or stubs. For example, in some embodiments, the compliant mechanism has pegs 150, and holes 152, such that a plurality of pegs mate with a plurality of holes to secure the components together. Pegs 150 and holes 152 may be configured such that components comprising the first joint member 102 (102a, 102b, 102c), and the second joint member 104(104a, 104b, 104C), are fixedly attached, but still allow the at least one flexure element to bend. In some embodiments, the shape of pegs 150 and/or holes 152 are round, square, oval, polygonal and any other shape known to one skilled in the art. In some embodiments, the components comprising the first joint member 102, and the second joint member 104 may be welded together (including e-beam or laser welding).

In some embodiments, first flexure element 106, second flexure element 107 and third flexure element 108 may function as springs and intersect independent of one another to provide an axis of rotation for first joint member 102 and second joint member 104. In some embodiments, first flexure element 106, second flexure element 107 and third flexure element 108 may intersect at any point along their length. In some embodiments, the center of rotation may be approximated by the intersection point, but moves as the flexure elements deform. In some embodiments, flexure elements may have different lengths and can be oriented at any angle, wherein changing any of these properties affects the mechanical behavior of the mechanism. In some embodiments, flexure elements may be oriented such that the first flexure element 106, second flexure element 107, and third flexure element 108 form a crossing pattern. In some embodiments, flexure elements may be oriented such that the first flexure element 106, second flexure element 107, and third flexure element 108 are substantially parallel.

In any of the embodiments described herein, the joint members may be made from any material known to one skilled in the art including but not limited to pure metals, metal alloys, polymers, ceramics, metallic glasses, or combinations thereof. In some embodiments, the joint members may be made from a biocompatible material. In some embodiments, the joint members may be coated in a biocompatible material. In some embodiments, the joint members may comprise a titanium alloy including but not limited to Ti6-Al4v. In some embodiments, the joint members may comprise Cr-Co. In some embodiments, the joint members may comprise a stainless steel including but not limited to SS316. In some embodiments, the joint members may comprise polyethene or ultra-high molecular weight polyethylene.

In some embodiments, device 100 is configured such that first flexure element 106, second flexure element 107 and third flexure element 108 are in tension when the mechanism is in compression.

In any embodiments described herein, the flexure elements may be made from any material known to one skilled in the art including but not limited to pure metals, metal alloys, polymers, ceramics, metallic glasses, or combinations thereof. In some embodiments, the flexure elements may comprise a biocompatible material. In some embodiments, the flexure elements may be coated in a biocompatible material. In some embodiments, the flexure elements may comprise Ti6-Al4v. In some embodiments, the flexure elements may comprise Cr-Co. In some embodiments, the flexure elements may comprise SS316.

In some embodiments, device 100 is configured to allow high compressive loads. In some embodiments, device 100 is configured to undergo a load ranging approximately between 10-30000 N.

Proximal anchor or tissue anchor 118 is attached to first joint member 102 at one end and is configured to attach device 100 to bone at the opposing end. Distal anchor or tissue anchor 120 is attached to second joint member 104 at one end and is configured to attach device 100 to bone at the opposing end. In some embodiments, device 100 may be anchored to the surrounding bones through proximal anchor 118 and distal anchor 120 via any method known to one skilled in the art including but not limited to intramedullary fixation, extramedullary fixation, surface osseointegration, etc. In some embodiments, any of a number of strategies for intramedullary fixation may be used to anchor device to the surrounding bone including but not limited to: long stem, short stem, threaded intramedullary screws, press-fit or cemented stems, nails, tapped and threaded implants, compression-style implants, porous coated for bone ingrowth, tibial trays, etc. In some embodiments, intraosseous rods or pins may be used for fixation to cancellous bone. In some embodiments, portions of any disclosed device may be formed as a tibial tray and/or tibial anchor. For example, first joint member 102 or second joint member 104 may be at least partially formed in the shape of a tibial tray, wherein a tissue anchor extends out from or is connected to the tibial tray. In some embodiments, the joint members are connected or fixedly attached to the tibial tray either directly or through additional interconnecting component parts.

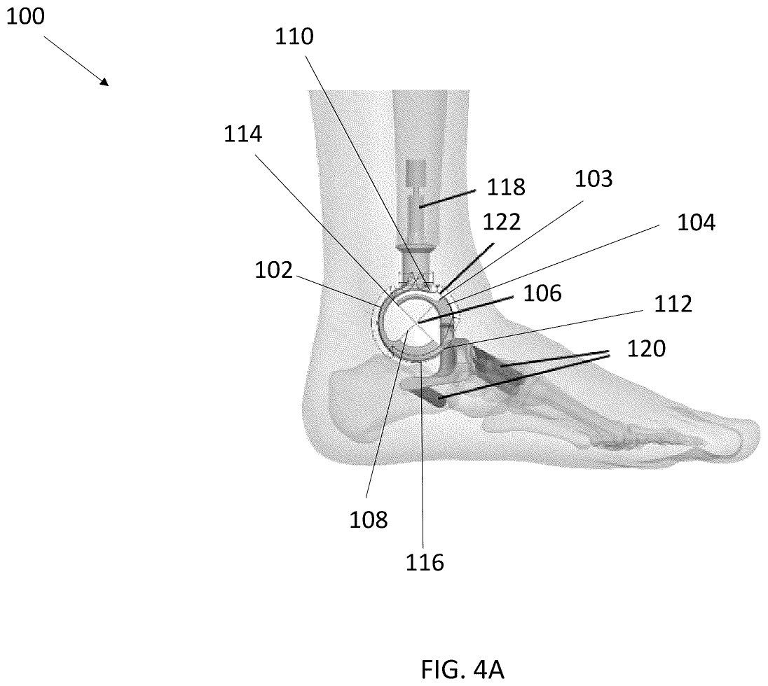

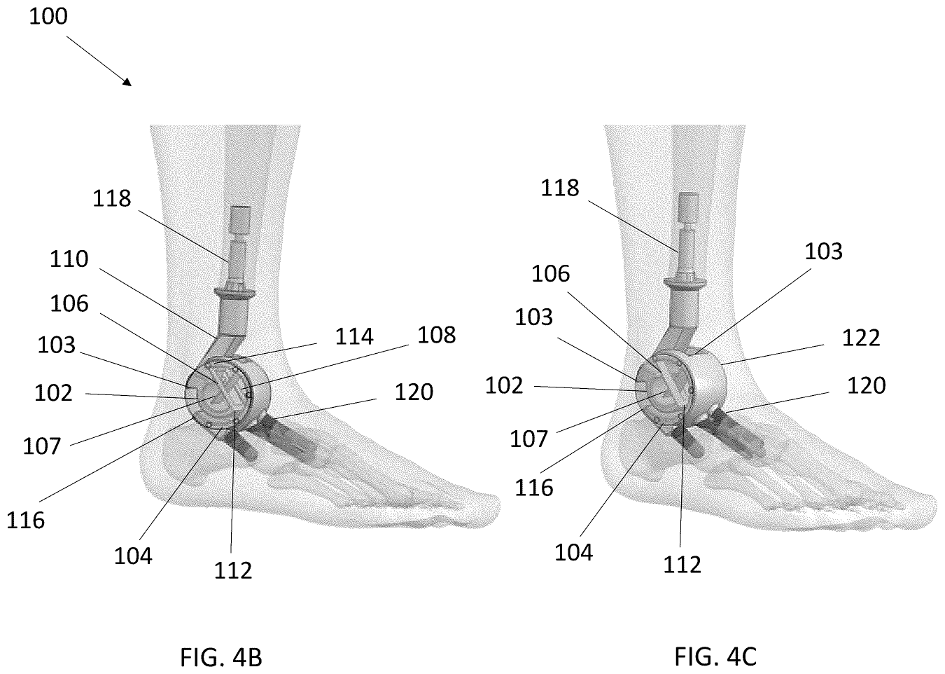

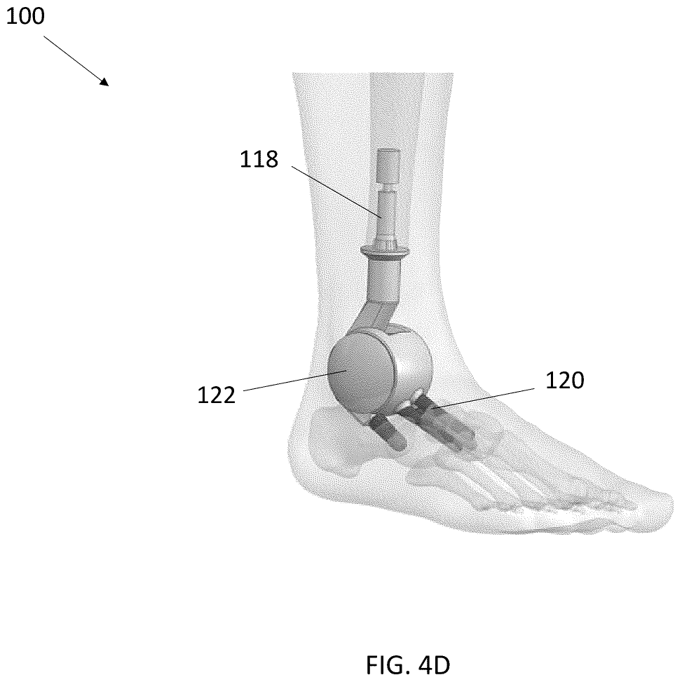

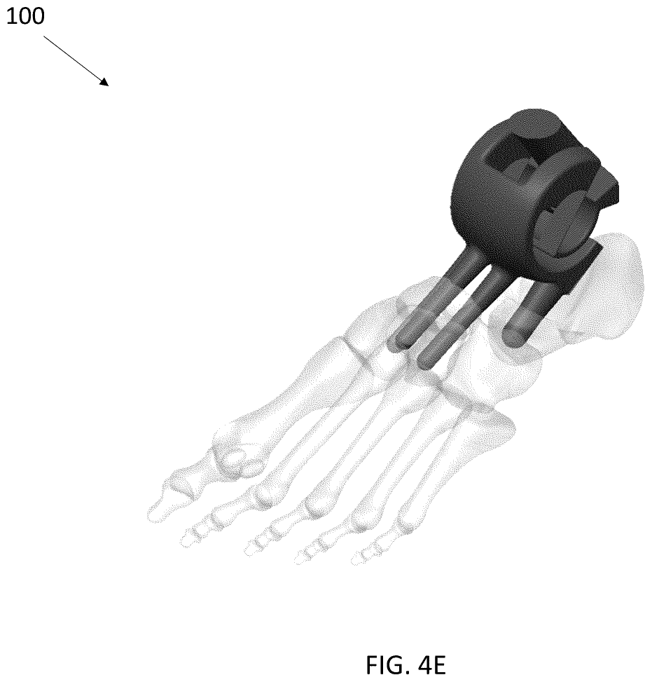

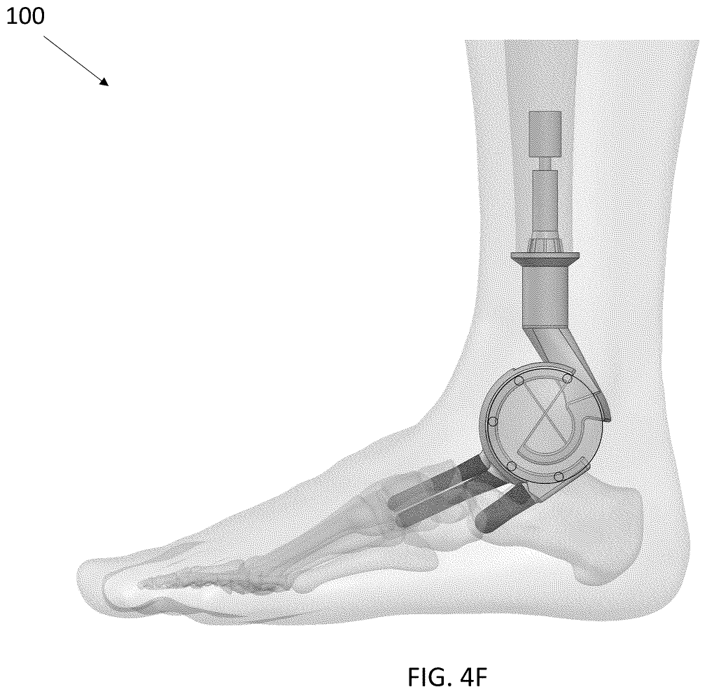

Referring now to FIG. 4A through FIG. 4F is a device 100 anchored to surrounding bone through proximal anchor 118 and distal anchor 120 by intramedullary fixation. In some embodiments, device 100 comprises a first joint member 102, a second joint member 104, at least one flexure element, a proximal anchor 118, at least one distal anchor 120, and a cover 122. Cover 122 is configured to at least partially enclose first joint member 102 and second joint member 104 and is configured to protect the surrounding soft tissues. In some embodiments, cover 122 may comprise a small gap that would allow fluids to pass through inside. In some embodiments, cover 122 may have plurality of pores to allow fluid to pass through inside to first joint member 102 and second joint member 104. In some embodiments, cover 122 may be made from any material known to one skilled in the art including but not limited to pure metals, metal alloys, polymers, ceramics, metallic glasses, or combinations thereof. In some embodiments, cover 122 may comprise a biocompatible material. In some embodiments, cover 122 may be coated in a biocompatible material. In some embodiments, cover 122 may comprise Ti6-Al4v. In some embodiments, cover 122 may comprise Cr-Co. In some embodiments, both devices may comprise SS316.

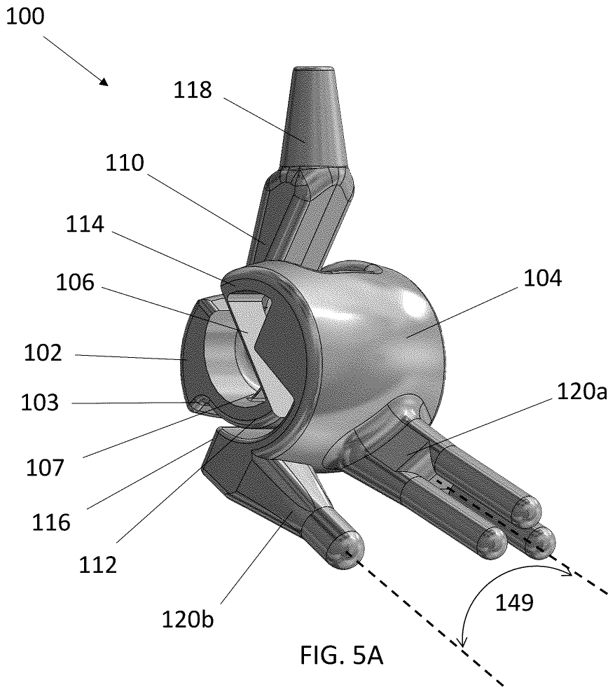

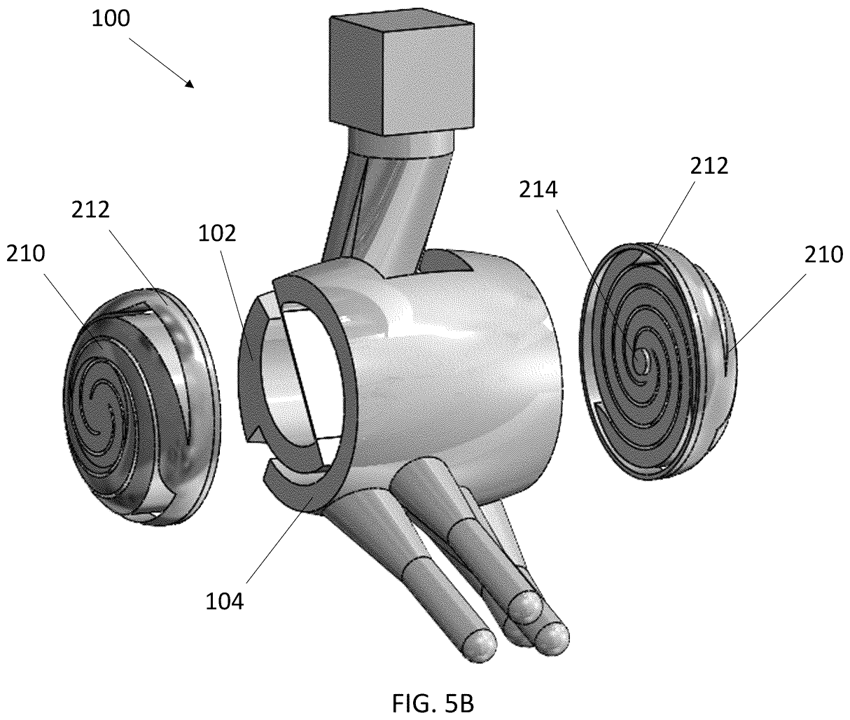

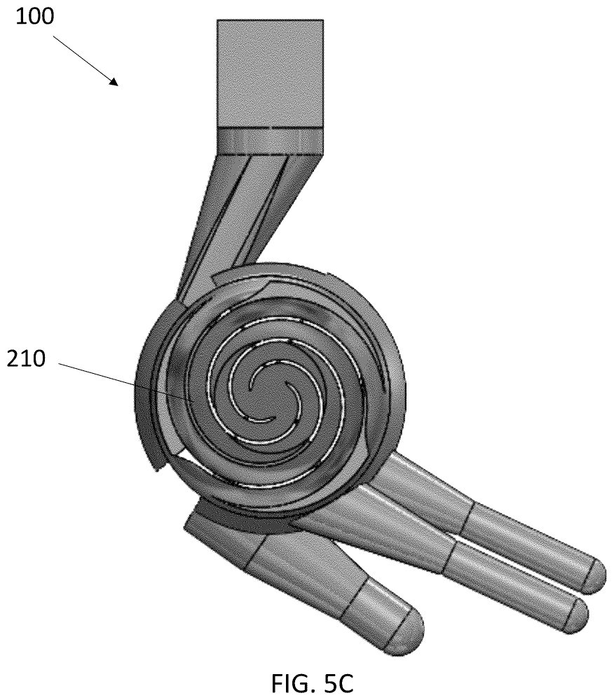

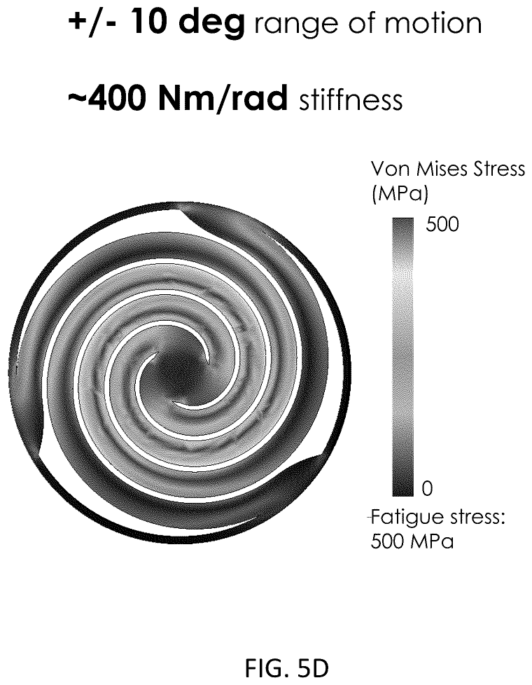

Referring now to FIG. 5A through 5B depicts of an exemplary undeformed implant device 100 of the present invention similar to that shown in FIGS. 3A and 3B and utilizes the same numbered parts for simplicity of description.

In some embodiments, device 100 further comprises a first distal anchor 120a and a second distal anchor 120b. In some embodiments, first distal anchor 120a has 3 pegs or rods protruding on its distal end. In some embodiments, the 3 rods of a first distal anchor 120a are meant to implant into three of the cuneiform bones of the foot. In some embodiments, first distal anchor 120a and second distal anchor 120b have a relative angle 149. In some embodiments, angle 149 is at least 5 degrees. In some embodiments, angle 149 is at least 10 degrees. In some embodiments, angle 149 is at least 15 degrees.

In some embodiments, angle 149 is at least 20 degrees. In some embodiments, angle 149 is at least 25 degrees. In some embodiments, angle 149 is between the range of 5 degrees and 40 degrees. In some embodiments, angle 149 is at least 10 degrees and 30 degrees. In some embodiments, first distal anchor 120a and second distal anchor 120b are divergent in multiple planes. In some embodiments, first distal anchor 120a and/or second distal anchor 120b are threaded screws that may be fixedly attached to the distal end of device 100. In some embodiments, first distal anchor 120a comprises pegs or rods placed in the midfoot bones (cuneiforms, navicular). In some embodiments, these pegs or rods can be cemented, porous coated, press fit, screwed, etc.

In some embodiments, at least one coil spring 210 may be used in parallel with device 100 (FIG. 5B and FIG. 5C). In some embodiments, coil spring 210 has a first joint member 212, a second joint member 204, and at least one flexure element connecting the first and second joint member. In some embodiments, coil spring 220 has rotational flexing with at least +/−10 degrees of motion. In some embodiments coil spring 220 has between a 5 and 10 degree range of motion. In some embodiments coil spring 220 has between a 10 and 15 degree range of motion. In some embodiments coil spring 220 has between a 20 and 25 degree range of motion. In some embodiments coil spring 220 has between a 25 and 30 degree range of motion. In some embodiments coil spring 220 has between a 30 and 45 degree range of motion. In some embodiments, first joint member 212 may be attached to second joint member 104 and second joint member 214 may be attached to first joint member 102, such that translational movement of the members causes a rotational deformation of coil spring 210. In some embodiments, first joint member 212 may be attached to first joint member 102 and second joint member 214 may be attached to the second joint member 104. In further embodiments, this could also be achieved with linear springs placed between the mechanism and each hard-stop, wherein one spring is to guide motion and the second spring is to store and return energy.

In some embodiments, the second spring may have any shape known to one skilled in the art including but not limited to coil spring, plurality of linear springs, cross-axis pivot, etc.

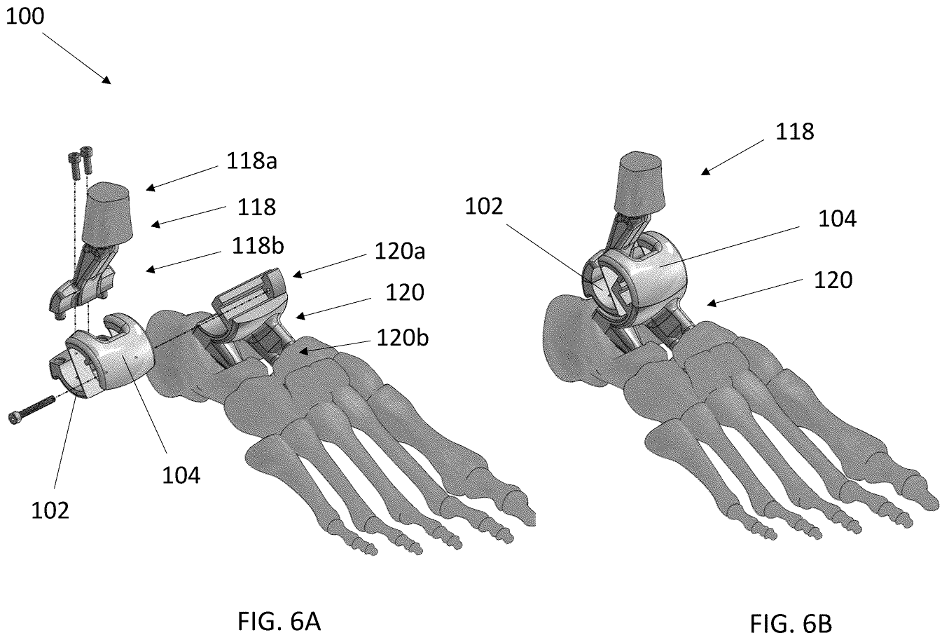

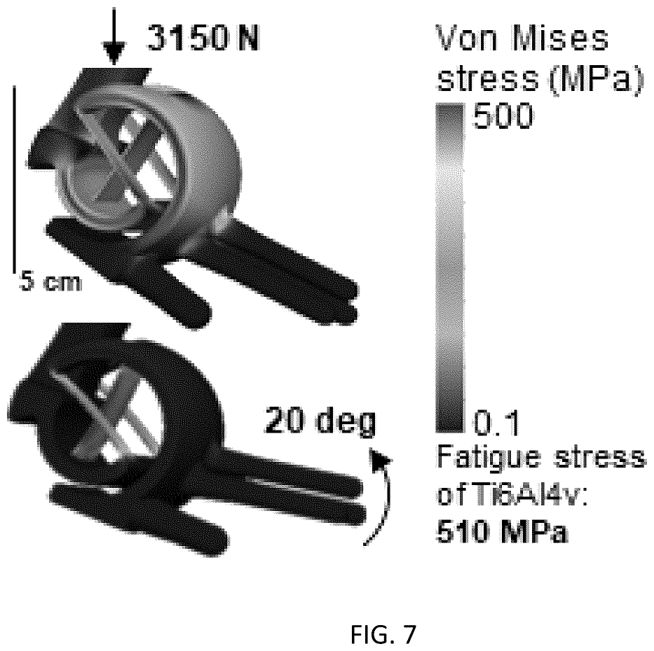

Aspects of the invention relate to a modular implant device for appropriate sizing and fitting a device to a subject. A modularity for the implant device also aids in the implantation of the device and allows for possible reparative or revision procedures without disturbing the surrounding bone. Referring now to FIG. 6A through FIG. 6B depicts an exemplary modular implant device 100 of the present invention. FIG. 6A depicts an exploded perspective view of an exemplary modular implant device 100 wherein the proximal anchor, flexure element and distal anchor are separate components that are fixedly attached or connected together during a surgical procedure. FIG. 6B depicts a perspective view of an assembled modular implant device 100 of the present invention applied to the ankle joint. In some embodiments, proximal anchor 118 and distal anchor 120 are separate components but may be fixedly attached or connected to first joint member 102, and second joint member 104, respectively. The separate components may be fixedly attached or connected by any method known to one skilled in the art including but not limited to, adhesives, cements, glues, friction fit, screws, bolts, threads, slots, tongue & groove, pegs, holes, etc.

Each anchor has a proximal region that engages with at least one joint member, and a distal region which engages with bone or tissue. In some embodiments, proximal anchor 118 comprises a proximal engagement region 118a for engaging with first joint member 102, and distal engagement region 118b for engaging bone or other tissue. In some embodiments, distal anchor 120 comprises a proximal engagement region 120a for engaging with second joint member 104, and distal engagement region 120b for engaging bone or other tissue.

In one exemplary embodiment, distal anchor 120 may be attached to calcaneus, navicular, and cuneiform bones when device 100 is used in an ankle-hindfoot joint.

In some embodiments, device 100 may be anchored to surrounding bone through proximal anchor 118 and distal anchor 120 by extramedullary fixation. In some embodiments, any of a number of strategies for intramedullary fixation may be used to anchor device to the surrounding bone including but not limited to: plates and screws. In some embodiments, conventional orthopedic hardware may be used for extra-osseous fixation including but not limited to customized plates and screws, standardized plates and screws, etc. In some embodiments, device 100 may be anchored to surrounding bone by ingrowth on a porous surface. In some embodiments, bony surfaces may be prepared intraoperatively for ingrowth onto a porous surface (e.g., porous coating for bone ingrowth as in hip replacement acetabular cups). In some embodiments, initial stability may be aided by combination with any other fixation strategy.

In some embodiments, proximal anchor 118 and distal anchor 120 may have any shapes/geometries known to one skilled in the art including but not limited to cylindrical, cubical, etc.

In some embodiments, proximal anchor 118 and distal anchor 120 may be made from any material including but not limited to titanium, Ti6-A14v, Cr-Co, SS316. In some embodiments, proximal anchor 118 and distal anchor 120 may be porous. In some embodiments, proximal anchor 118 and distal anchor 120 may be further coated with any material known to one skilled in the art including but not limited to pure metals, metal alloys, polymers, ceramics, metallic glasses, or combinations thereof. In some embodiments, proximal anchor 118 and distal anchor 120 may comprise titanium. In some embodiments, proximal anchor 118 and distal anchor 120 may comprise a biocompatible material.

In some embodiments, device 100 may be used for common orthopedic procedures including but not limited to total hip replacement, total knee replacement, total wrist replacement, total ankle replacement, total shoulder replacement, etc. In some embodiments, it is possible to surgically alter the joint (e.g., foot) to facilitate fixation. In one exemplary embodiment, joints may be fused to eliminate motion that would be deleterious to bony ingrowth and osseointegration. In some embodiments, bones may be fused together to create larger bony surfaces to which the implant could be fixed. In some embodiments, bones may be removed or relocated to create more space for the implant.

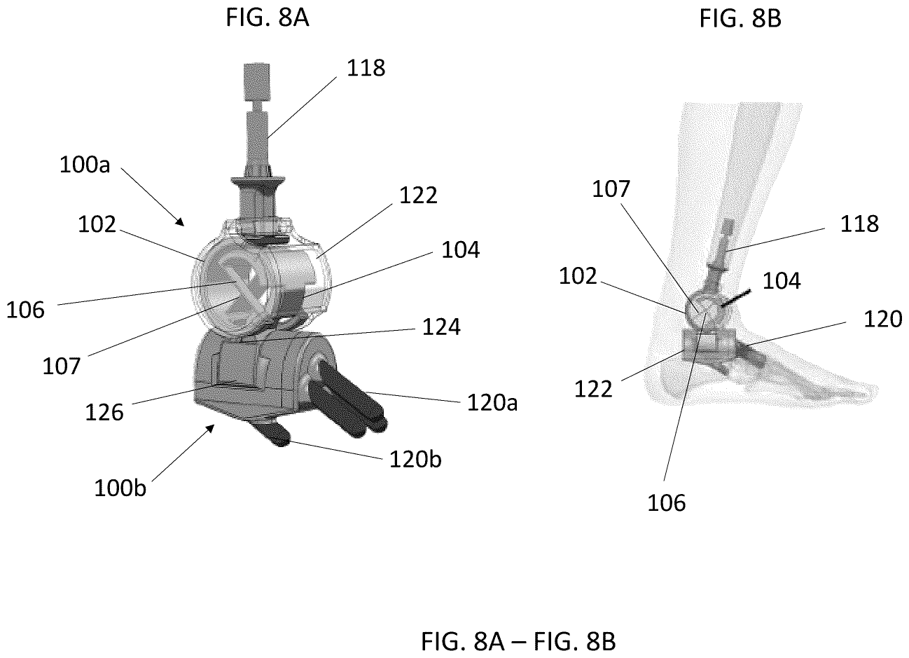

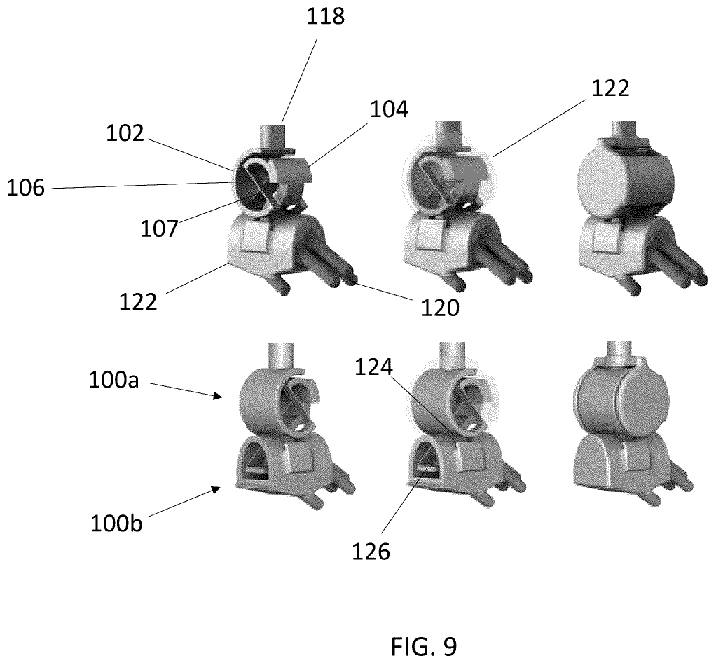

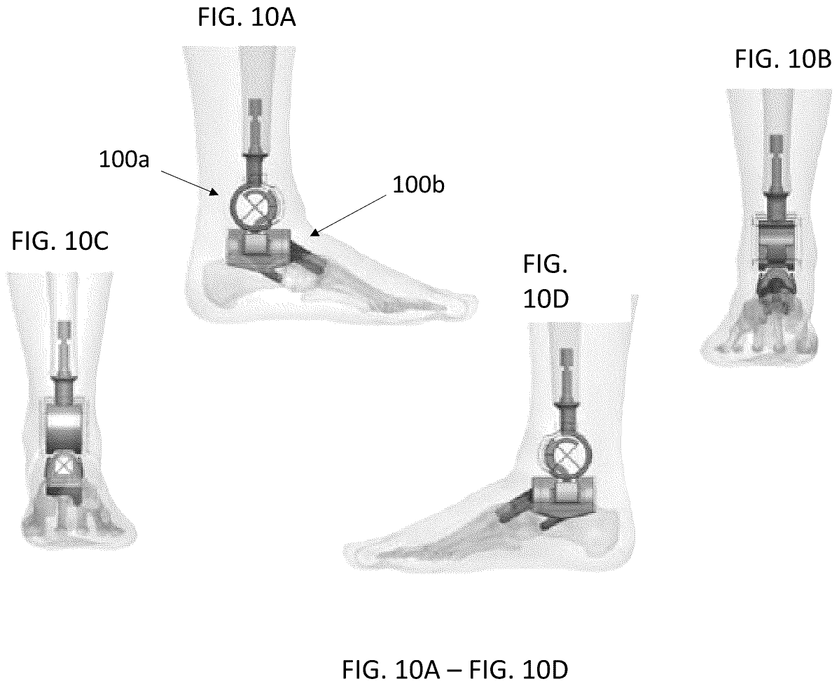

Referring now to FIG. 8A, in some embodiments, an implant may comprise a first compliant mechanism in series with a second compliant mechanism to create at least a 2 degree of freedom system. In some embodiments, devices of the present invention may be combined in series with a second compliant mechanism to create a 2 degree of freedom in an ankle and a subtalar joint (FIG. 8B). In some embodiments, the second degree of freedom may be a second cross-axis pivot in tension. In some embodiments, the second degree of freedom may be any other mechanism known to one skilled in the art. In some embodiments, the implant comprises a first compliant device 100a, which can be similar to device 100 as described and utilizes the same numbered parts for simplicity of description. However, instead of being engaged with a distal anchor, device 100a engages a second compliant device 100b, which can be similar to device 100 as described and utilizes the same numbered parts for simplicity of description. Device 100b permits similarly to 100a, a degree of freedom, however this degree of freedom is on another plane allowing a different range of motion or movement. In some embodiments, device 100b replaces distal anchor 118 of device 100a. In some embodiments, bottom region 116 of device 100a connects with top region 110 of device 100b.

In some embodiments, device 100 may further comprise a third joint member 124 having an inner surface and an outer surface, a fourth joint member 126 having an inner surface and an outer surface, at least one flexure element having first and second ends, wherein the first end is connected to the inner surface of third joint member 124 and the second end is connected to the inner surface of fourth joint member 126, wherein third joint member 124 and fourth joint member 126 are configured to move relative to each other within a second degree of freedom that is different than the first degree of freedom of movement between first joint member 102 and second joint member 104, and wherein the outer surface of third joint member 124 is connected to the outer surface of second joint member 104. In some embodiments, the first and second degrees of freedom are each translational. In some embodiments, the first degree of freedom is translational and the second degree of freedom is rotational. In some embodiments, the first and second degrees of freedom are each rotational.

In some embodiments, the device further comprises a proximal tissue anchor (e.g. proximal anchor 118) extending from the outer surface of first joint member 102, and at least one distal tissue anchor (e.g. first distal anchor 120a, second distal anchor 120b) extending from the outer surface of fourth joint member 126. In some embodiments, the tissue anchors are each configured to engage bone via at least one selected from the group consisting of intramedullary fixation, extramedullary fixation, osseointegration and combinations thereof.

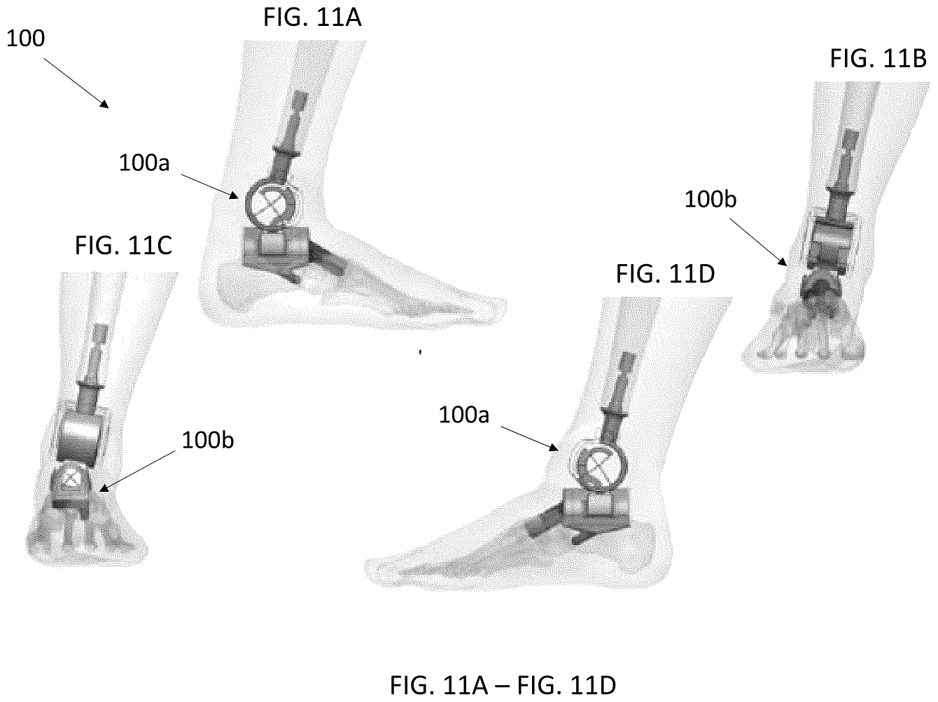

Referring now to FIG. 11A and FIG. 11D, movement for compliant device 100a applied to an ankle joint is shown. Referring to FIG. 11C and 11B, movement for compliant mechanism 100b applied to an ankle joint is shown.

In some embodiments, device 100 may be used in the ankle joint. In some embodiments, device 100 may be used to replace any joint in the body, including but not limited to joints of fingers, thumb, hand, wrist, elbow, shoulder, toes, foot, ankle/subtalar, knee, hip and etc. In some embodiments, device 100 may be used to replace spinal vertebrae in the back or neck.

In some embodiments, device 100 may be used in rehabilitative applications. In some embodiments, device 100 may be used in preventive/augmentative applications, in which no pathology has been diagnosed, and the purpose is to improve upon baseline function or prevent future pathology. In some embodiments, device 100 may be used for treating a subject having a diagnosed pathology. In some embodiments, device 100 may be used for a subject seeking prophylactic injury prevention. In some embodiments, device 100 may be used for a subject to enhance movement ability beyond their baseline capacity.

In some embodiments, device 100 may be used in parallel with existing joints, to manipulate their mechanical properties and provide energy storage and return. In one exemplary embodiment, device 100 may be implanted in parallel with the joints of a subject with joint weakness, to generate a restorative torque about the joint, provide energy storage, and enable improved function.

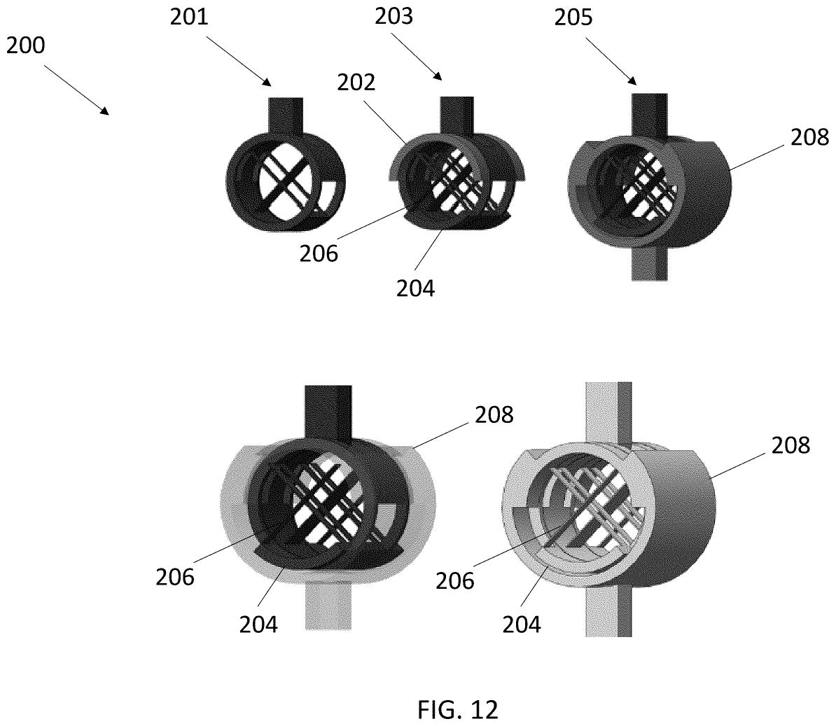

Referring now to FIG. 12, another exemplary implant device 200 is shown.

In some embodiments, device 200 comprises a first stage element 201, two second stage elements 203 and a third stage element 205. First stage element 201 is structurally and functionally similar to device 100 as described elsewhere herein.

Second stage elements 203 comprise a first joint member 202, a second joint member 204, and a pair of crossing flexure elements 206. In some embodiments, first joint member 202 and second joint member 204 may have any shape known to one skilled in the art. In some embodiments, first joint member 202 and second joint member 204 may have a circular or rounded outer geometry. In some embodiments, first joint member 202 and second joint member 204 may be made from any material known to one skilled in the art including but not limited to pure metals, metal alloys, polymers, ceramics, metallic glasses, or combinations thereof. In some embodiments, first joint member 202 and second joint member 204 may comprise a biocompatible material. In some embodiments, first joint member 202 and second joint member 204 may be coated in a biocompatible material. In some embodiments, first joint member 202 and second joint member 204 may comprise Ti6-A14v. In some embodiments, first joint member 202 and second joint member 204 may comprise Cr-Co. In some embodiments, first joint member 202 and second joint member 204 may comprise SS316.

A pair of flexure elements 206 connect first joint member 202 to second joint member 204, such that one end of the pair of flexure elements 206 is attached to first joint member 202 and the other end is connected to second joint member 204. A pair of flexure elements 206 may have any thickness known to one skilled in the art. In some embodiments, a pair of flexure elements 206 may have the same thickness. In some embodiments, a pair of flexure elements 206 may have different thicknesses. In some embodiments, a pair of flexure elements 206 may have a thickness ranging between 0.005-5 mm. In some embodiments, a pair of flexure elements 206 may have varying thicknesses along their length. In some embodiments, a pair of flexure elements 206 may have any width known to one skilled in the art. In some embodiments, a pair of flexure elements 206 may have the same width. In some embodiments, a pair of flexure elements 206 may have different widths. In some embodiments, a pair of flexure elements 206 may have a width ranging between 0.1-10 cm. In some embodiments, a pair of flexure element 206 may have varying widths along their length. In some embodiments, a pair of flexure elements 206 may have any length known to one skilled in the art. In some embodiments, a pair of flexure elements 206 may have the same length. In some embodiments, a pair of flexure elements 206 may have different lengths. In some embodiments, a pair of flexure elements 206 may have a length ranging between 0.2-20 cm.

In some embodiments, thickness, width, and length of a pair of flexure elements 206 may be modified to change overall behavior including but not limited to range of motion, load bearing capacity, stiffness, center of rotation trajectory, etc.

First stage element 201 and two second stage elements 203 are nested in series such that two second stage elements 203 are positioned on either side of first stage element 201. In some embodiments, first stage element 201 and two second stage elements 203 are configured such that all flexure elements are in tension when the mechanism is in compression.

Third stage element 205 comprises a shell 208 and is positioned around the perimeter of first stage element 201 and two second stage elements 205. Shell 208 has a diameter ranging between approximately 0.5 cm and 15 cm.

In some embodiments, device 200 is configured to provide more ranges of motion compared to device 100. In some embodiments, device 200 provides at least one degree of freedom. In some embodiments, device 200 may be used in series with other mechanisms to add more degrees of freedom.

In some embodiments, device 200 may be used in the knee joint. In some embodiments, device 200 may be used in the elbow joint. In some embodiments, device may be used to replace any joint in the body, including but not limited to joints of fingers, thumb, hand, wrist, shoulder, toes, foot, ankle/subtalar, hip and etc. In some embodiments, device may be used to replace spinal vertebrae in the back or neck.

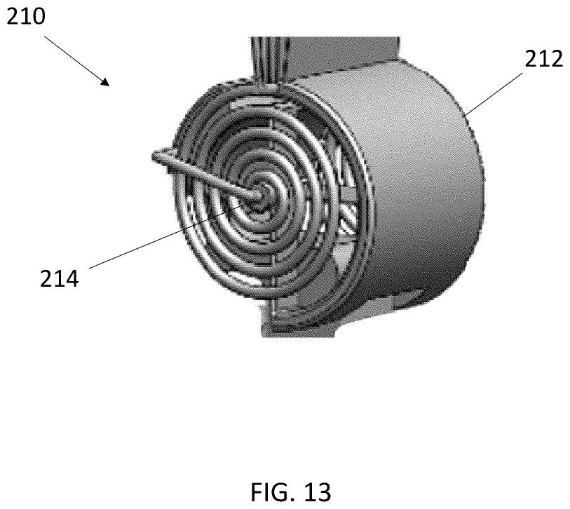

In one exemplary embodiment, a coil spring 210 may be used in parallel with both device 100 and device 200 (FIG. 13). Coil spring 210 comprises an outer edge 212 and an inner point 214. In some embodiments, outer edge 212 may be attached to first stage element 201 and inner point 214 may be attached to second stage elements 203, such that rotation of the compliant joint cause deformation of coil spring 210. In some embodiments, this could also be achieved with linear springs placed between the mechanism and each hard-stop, wherein one spring is to guide motion and the second spring is to store and return energy. In some embodiments, the second spring may have any shape known to one skilled in the art including but not limited to coil spring, plurality of linear springs, cross-axis pivot, etc. In some embodiments, outer edge 212 may be attached to first joint member 102 and inner point 214 may be attached to the second joint member 104.

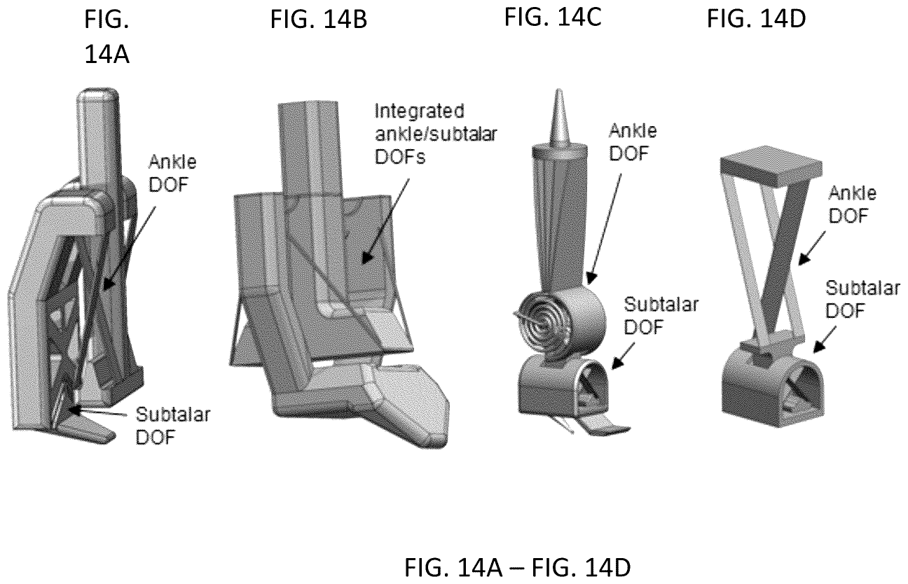

Referring now to FIG. 14A through FIG. 14D, additional exemplary compliant mechanism architectures are shown. In some embodiments, any compliant architecture may be used to replace a biological joint including but not limited to: a cross-axis pivot (FIG. 14A, FIG. 14C, FIG. 14D), suspended wire flexures (FIG. 14B), intersecting leaf springs (FIG. 14A, FIG. 14C), etc. Unlike the other embodiments (FIG. 14A, FIG. 14C, FIG. 14D), which achieve two skew orthogonal rotational degrees of freedom (DOFs) by stacking two parallel joints in series, the suspended wire-flexure embodiment (FIG. 14B) achieves two desired DOFs by directly joining two rigid bodies together within a single parallel joint using a plurality of wire flexures. The embodiment depicted in FIG. 14B comprises four wire flexures, wherein the axes of both rotational

DOFs intersect the axes of the four wire flexures to achieve the desired skew orthogonal rotations. Single rotational DOFs can also be achieved using two or more blade flexures or leaf springs that lie on planes that intersect along the axis of rotation, which is depicted in the lower portion of FIG. 14A labeled Subtalar DOF and in the upper half of FIG. 14C to enhance rotational range of motion of the DOF labeled Ankle DOF. In some embodiments, at least one compliant mechanism may be used in replacing one joint. In some embodiments, compliant mechanisms with different architectures may be used in replacing one joint. In some embodiments, compliant mechanism may provide at least one degree of freedom.

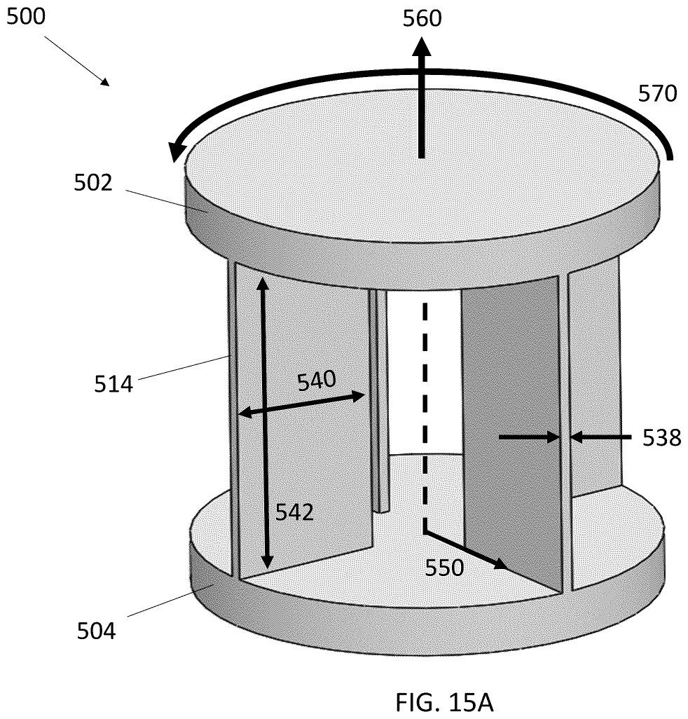

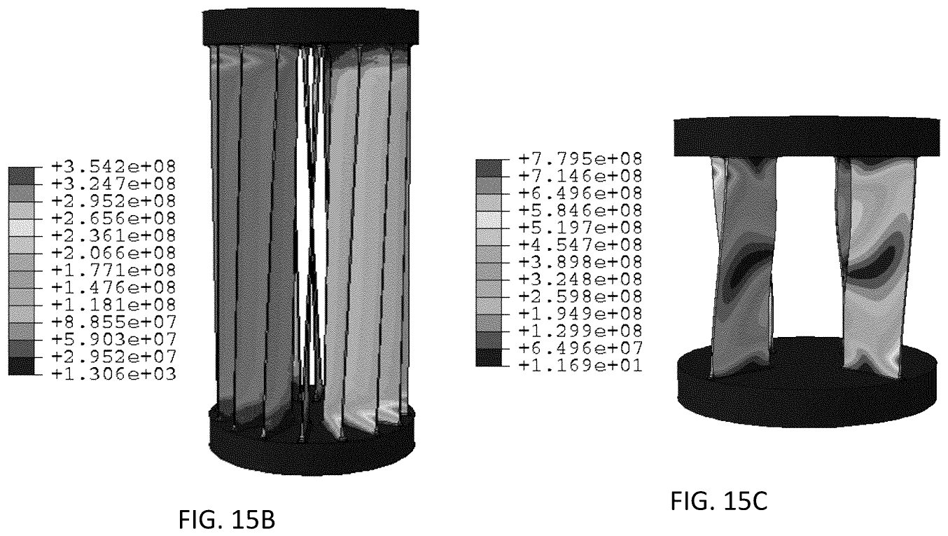

Referring now to FIG. 15A, an exemplary implant device 500 is shown. In some embodiments, device 500 comprises a first joint member 502, a second joint member 504 connected by at least one flexure element. In some embodiments, first joint member 502 and second joint member 504 may have any shape known to one skilled in the art. In some embodiments, first joint member 502 and second joint member 504 may have a circular or rounded outer geometry to allow first joint member 502 and second joint member 504 to rotate cleanly about a center of rotation. In some embodiments, first joint member 502 has a larger diameter than second joint member 504.

In some embodiments, the at least one flexure element comprises at least one element or blade, or a plurality of blades 514, wherein the element or blade has a thickness 538, a width 540, a length 542, and a distance to center of blade 550. In some embodiments, device 500 may experience a tensile load 560 and a rotational load 570. In some embodiments, plurality of blades 514 may have a length 542 ranging between about 0.3 cm to about 20 cm. In some embodiments, plurality of blades 514 may have a width 540 ranging between about 0.1 cm to about 3 cm. In some embodiments, plurality of blades 514 may have a thickness 538 of up to about 0.3 cm. In some embodiments, plurality of blades 514 may have a distance to center of blade 550 ranging between 0.1 cm and 20 cm. In some embodiments, plurality of blades 514 may be configured for compressive loads ranging from 1-10000 N. In some embodiments, plurality of blades 514 may be configured for tensile loads ranging from 1-10000 N. In some embodiments, plurality of blades 514 may be configured for rotational loads ranging from 1-10000 N*m. In some embodiments, plurality of blades 514 are arranged substantially parallel. In some embodiments, plurality of blades 514 are arranged concentrically. In some embodiments, plurality of blades 514 are arranged radially from a central axis. In some embodiments, plurality of blades 514 are equidistant about a central axis.

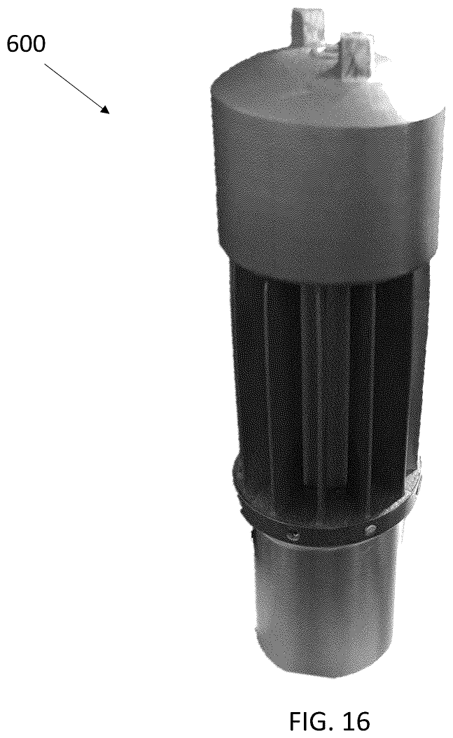

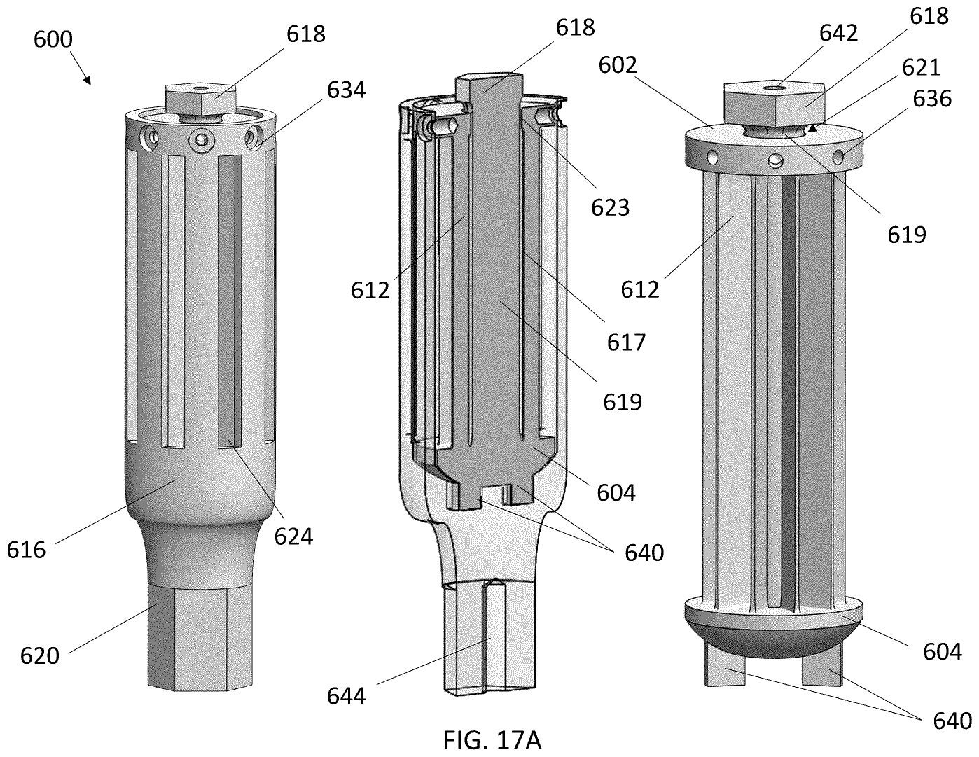

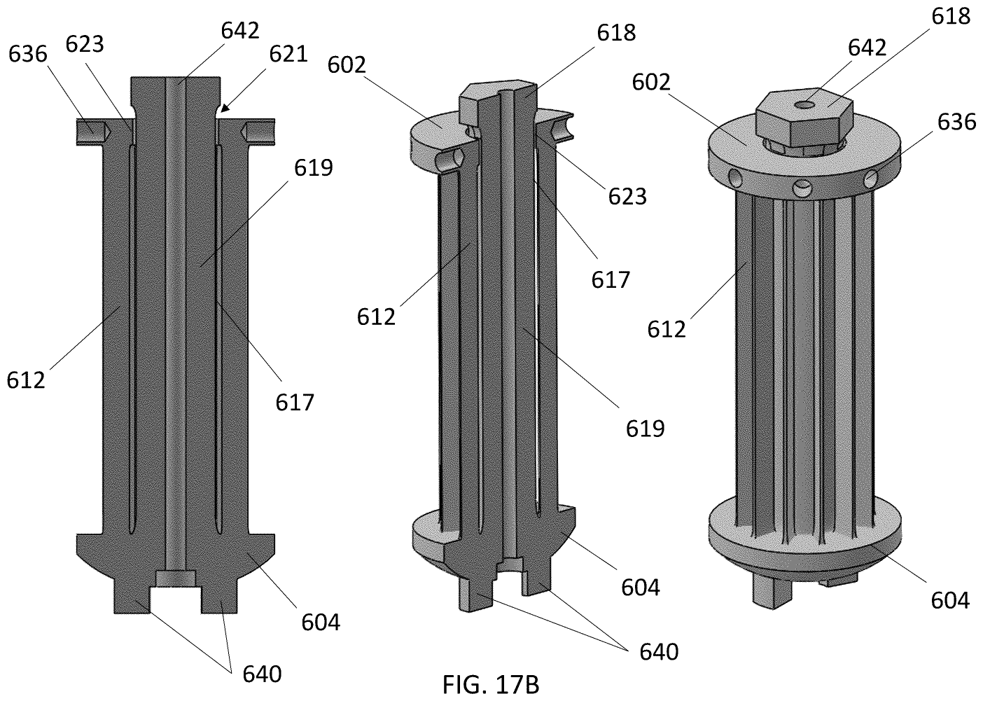

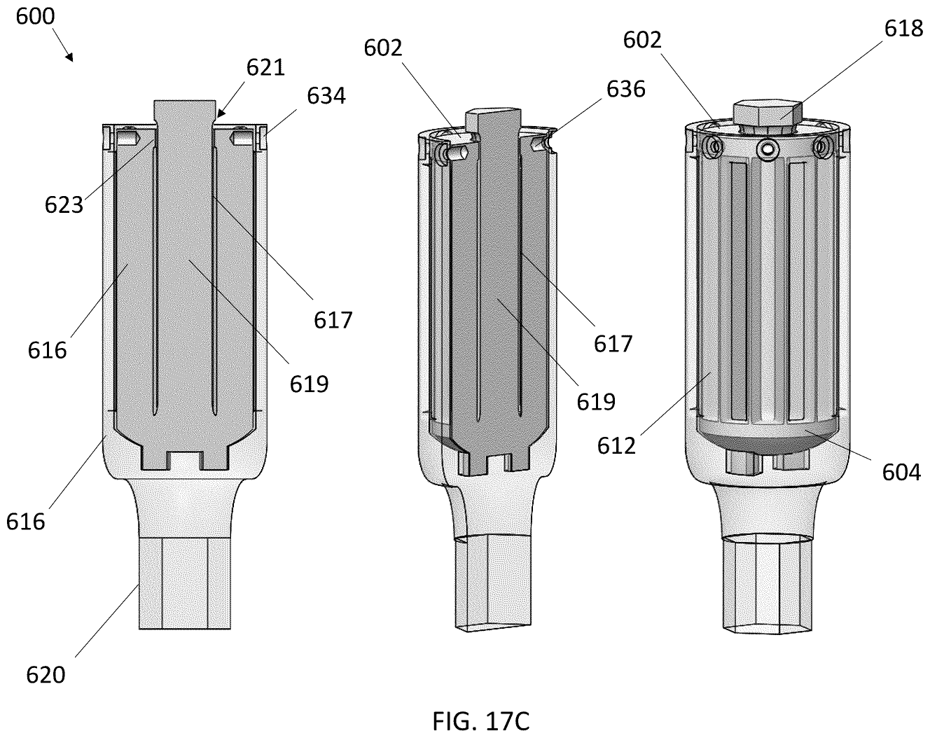

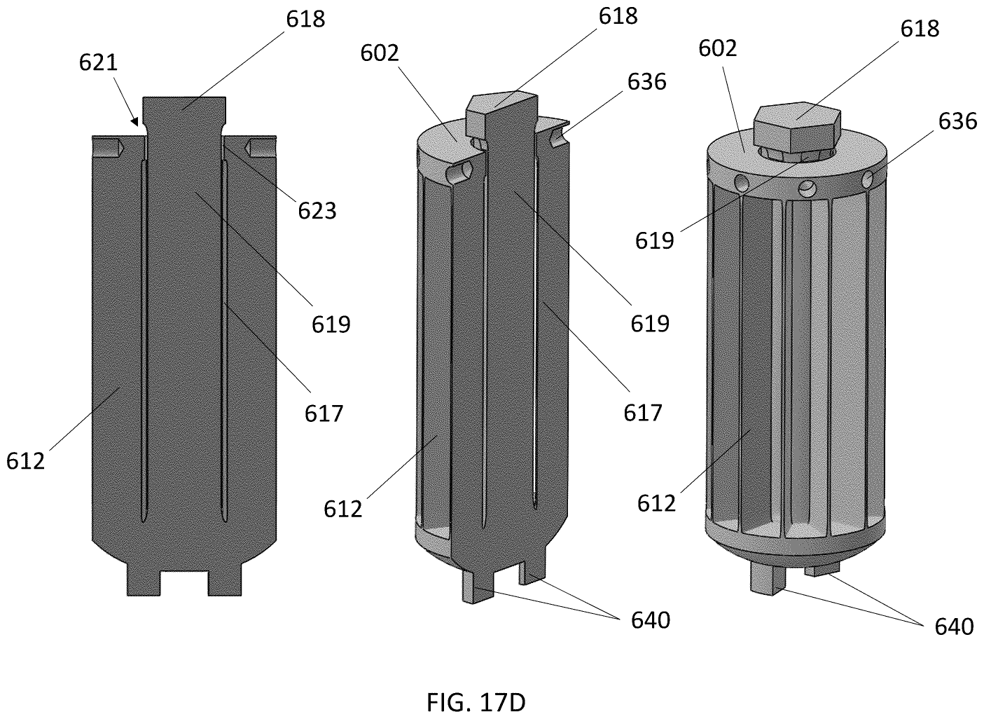

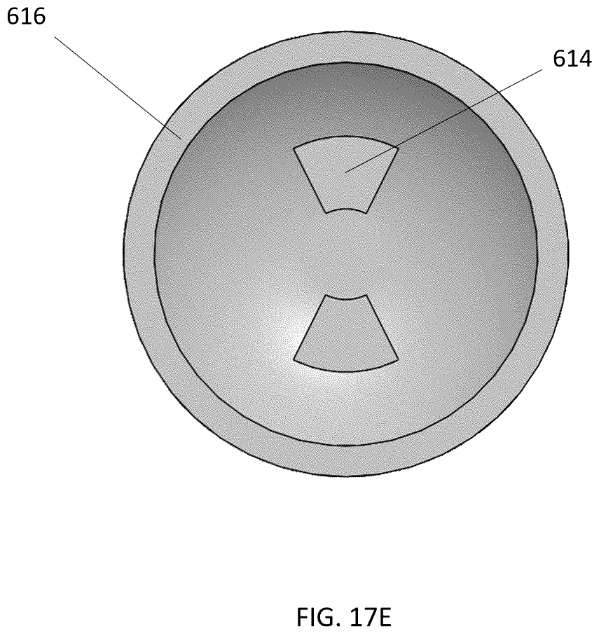

Now referring to FIGS. 16 and 17A through 17E, shown is an exemplary implant device 600 of the present invention that is inverted as compared to the embodiment of FIG. 15A. Like device 500, device 600 includes a first joint member 602, a second joint member 604 and a plurality of blades 612 connected to the first and second joint members 602 and 604.

In some embodiments, first joint member 602 and second joint member 604 may have any desired shape, as would be known by one skilled in the art. In some embodiments, first joint member 602 and second joint member 604 may have any desired thickness, for example ranging approximately between 0.05-10 mm. In some embodiments, first joint member 602 and second joint member 604 may have any desired width, or diameter, for example ranging approximately between 0.5-20 cm, depending on the joint. In some embodiments, width, thickness, and diameter of first joint member 602 and second joint member 604 may be modified to change overall behavior including but not limited to range of motion, load bearing capacity, stiffness, center of rotation trajectory, etc., depending on the type of joint being replaced. In some embodiments, first joint member 602 and second joint member 604 have variable width, thickness and/or diameter along their length. In some embodiments, first joint member 602 and second joint member 604 have uniform width, thickness and/or diameter along their length.

Similar to the embodiments of FIG. 15A, plurality of blades 612 may have a length ranging between about 0.3 cm to about 20 cm. In some embodiments, plurality of blades 612 may have a width ranging between about 0.1 cm to about 3 cm. In some embodiments, plurality of blades 612 may have a thickness of up to about 0.3 cm. In some embodiments, plurality of blades 612 may have a distance to center of blade ranging between 0.1 cm and 20 cm. In some embodiments, plurality of blades 612 may be configured for compressive loads ranging from 1-10000 N. In some embodiments, plurality of blades 612 may be configured for rotational loads ranging from 1-10000 N*m. In some embodiments, plurality of blades 612 are arranged substantially parallel. In some embodiments, plurality of blades 612 are arranged concentrically. In some embodiments, plurality of blades 612 are arranged radially from a central axis. In some embodiments, plurality of blades 612 are equidistant about a central axis.

Additionally, device 600 includes a central stem or post 619. In some embodiments, central post 619 is fixed to or otherwise contiguous with second joint member 604. In some embodiments, central post 619 is formed of a rigid material, a deformable material, a flexible material, or combinations thereof. In some embodiments, central post 619 is deformable about at least one axis (e.g., a central axis passing through first joint member 602 and second joint member 604) or allows at least one DOF. In some embodiments, central post 619 may deform or bend providing some compliance in bending about all axes perpendicular to the central axis. In some embodiments, first joint member 602 may include any shaped opening 621 such as a central, ring-shaped opening, which permits central post 619 to extend through opening 621 of first joint member 602. Accordingly, blades 612 are separate from post 619. In one embodiment, blades 612 are separated from central post 619 by gap 617. In some embodiments, gap 617 is contiguous with a space between the exterior surface of central post 619 and sidewall 623. In some embodiments, at least a portion of one or more blades may be in contact with central post 619. Importantly, first joint member 602 is not fixed to central post 619 such that first joint member 602 is able to rotate freely about central post 619. In some embodiments, opening 621 is sized and shaped to limit the range of deformation or movement of central post 619. Opening 621 comprises or forms one or more sidewalls 623 in first joint member 602 that may be configured as one or more range-limiting elements or stops for central post 619 during deformation. In some embodiments, opening 621 and/or one or more sidewalls 623 may be any suitable size or shape in order to limit and/or stop the deformation or movement of central post 619, and a result, the movement of second joint member 604. For example, but without limitation, opening 621 and/or one or more sidewalls 623 may be at least partially formed in any shape, such as a circle, a ring, a polygon, a square, an oval, or combinations thereof. In some embodiments, the one or more range limiting elements or stops of opening 621 limit the bending, deformation or movement of central post 619 in any and all axes perpendicular to the central axis.

Device 600 may also include a housing or outer shell 616 which surrounds the first and second joint members and the plurality of blades. Housing 616 may also be connected to or integrated with a distal anchor 620. In some embodiments, device 600 is configured such that distal anchor 620 and outer shell 616 are formed as a single unit. In some embodiments, distal anchor 620 can be separate from, or form a portion of housing 616. In some embodiments, outer shell 616 forms a cup with a proximal rim, with a cavity therein for receiving the compliant mechanism having at least one flexure element. In any embodiments described herein, the first and second joint members, the central post, the housing, anchor components, and plurality of blades may be made from any material known to one skilled in the art including but not limited to pure metals, metal alloys, polymers, ceramics, metallic glasses, and any coatings including porous coatings or combinations thereof. In some embodiments, the first and second joint members, the central post, the housing, anchor components, and plurality of blades may comprise a biocompatible material. In some embodiments, the first and second joint members, the central post, the housing, anchor components, and plurality of blades may be coated in a biocompatible material. In some embodiments, the first and second joint members, the central post, the housing, anchor components, and plurality of blades may comprise Ti6-Al4v. In some embodiments, the flexure elements may comprise Cr-Co. In some embodiments, the first and second joint members, the central post, the housing, anchor components, and plurality of blades may comprise SS316.

Referring again to FIG. 17A, an exploded side view of an implant device is shown. In some embodiments, device 600 is arranged such that distal anchor 620 and outer shell 616 are formed as a single unit. In some embodiments, outer shell 616 forms a cylindrical or cup shape with a proximal rim, with a cavity for receiving at least one compliant mechanism, such as first and second joint members 602 and 604 and the plurality of blades 612. Accordingly, the at least one compliant mechanism comprises a first joint member 602 having an inner and outer surface with a peripheral rim and at least one flexure element 612 and second joint member 604 having an inner and outer surface forming a single unit. In some embodiments, device 600 comprises shell set screw holes 634 arranged circumferentially around the proximal rim of housing or outer shell 616. In some embodiments, device 600 comprises top plate set screw holes 636 arranged circumferentially on the peripheral rim of first joint member 602. In some embodiments, top plate set screw holes 636 have threads for engaging at least one set screw. In this example, the at least one flexure element is inserted into the cup of outer shell 616 and fixedly attached to the proximal rim of the cup. To prevent movement, the housing is fixed to first joint member 602 using set screws positioned in shell set screw holes 634 and top plate set screw holes 636. Similar to device 300 described herein, the flexure elements of device 600 are in tension when the device is in compression.

As explained previously, device 600 further comprises an opening in the first joint member such that the first joint member forms a ring, and a central post having a first end fixed to or contiguous with the inner surface of the second joint member, and a second end extending through the opening of the first joint member such that the first joint member is rotatable around the post.

In some embodiments, device 600 further comprises a housing 616 surrounding at least the first joint member, the second joint member and the plurality of flexure elements, wherein the first joint member is fixed to the housing. In some embodiments, device 600 further comprises a first tissue anchor connected to the second end of the central post, and a second tissue anchor connected to the outer surface of the second joint member. In some embodiments, device 600 further comprises a first tissue anchor connected to the second end of the central post, and a second tissue anchor connected to the housing.

In some embodiments, the housing further comprises at least one recess, and the second joint member further comprises at least one tab extending from the outer surface of the second joint member and positioned within the at least one recess of the housing, wherein the at least one recess is sized to permit range-limiting movement of the at least one tab in the at least one recess when the second joint member is rotated relative to the first joint member.

In some embodiments, outer shell 616 comprises plurality of slits 624 allowing fluid communication between an inside and an outside of outer shell 616. In some embodiments, plurality of slits 624 are holes in the body of outer shell 616. In some embodiments, plurality of slits 624 may have a width ranging between about 0.1 mm to about 2 cm. In some embodiments, plurality of slits 624 may have a height ranging between about 0.1 mm to about 15 cm. In some embodiments, plurality of slits 624 may be any shape known to one skilled in the art.

In some embodiments, to further resist rotational movement, housing 616 of device 600 further comprises distal recesses or slots 614 within the cup of outer shell 616 for receiving distal tabs 640 when the implant device is assembled. In some embodiments, distal slots 614 are range limiting features allowing a constrained rotational path for second joint member 604. For example, tabs 640 may be sized to fit within slots 614 with limited movement. Then when second joint member 604 rotates inside housing 616, tabs 640 similarly rotate within slots 614 until tabs 640 come into contact with a wall of slot 614, at which point further rotational movement of second joint member 604 is restricted.

In some embodiments, device 600 comprises proximal anchor 618 having a top surface and distal anchor 620 having a bottom surface. In some embodiments, proximal mounting hole 642 is formed on the top surface of proximal anchor 618. In some embodiments, distal mounting hole 644 is formed on the bottom surface of distal anchor 620. These holes may be used, for example, in affixing device 600 to other components with screws, bolts, friction fits, etc. In some embodiments, device 600 may further comprise at least one hard stop or range-limiting element. For example, proximal anchor 618 may comprise range-limited features that contacts first joint member 602 to prevent damage from any force other than compressive force.

In some embodiments, device 600 may be inserted partially or fully into a bone. In some embodiments, at least a portion of device 600 is inserted into bone such that at least a portion of proximal anchor 618 protrudes from the bone.

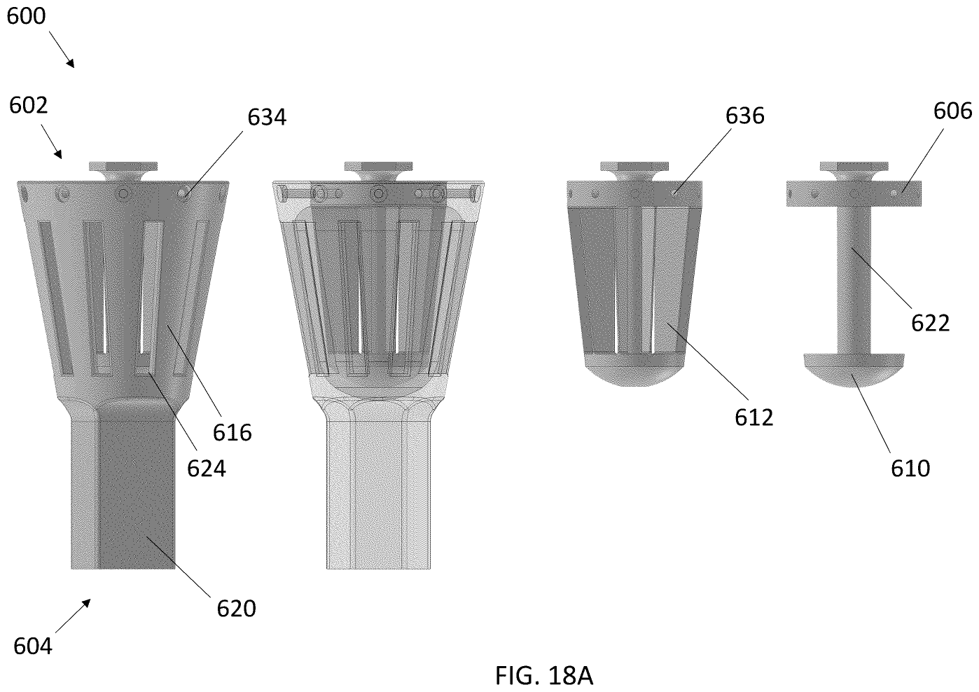

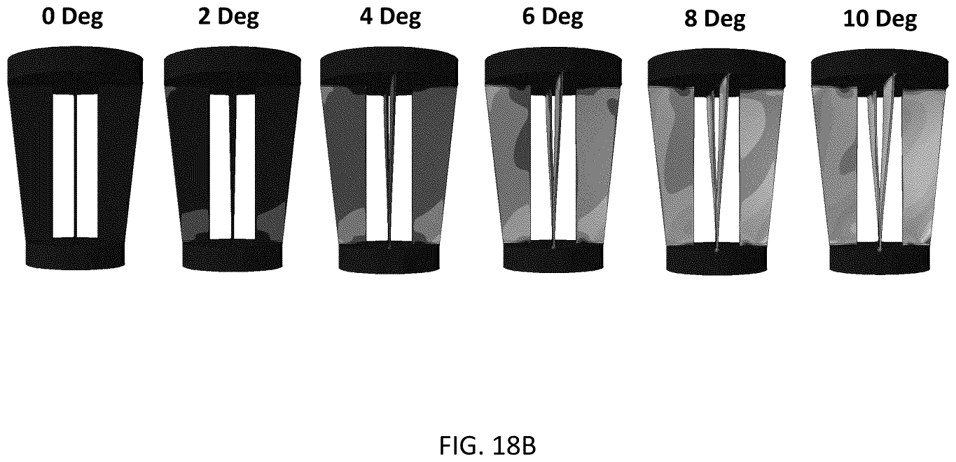

In some embodiments and as shown in FIG. 18A, the at least one flexure clement comprises a first joint member or top plate 606 with a peripheral rim and at least one flexure element 612 and a second joint member or bottom plate 610. Like the embodiments of FIG. 17A, a central stem or post 622 is fixed to or otherwise contiguous with bottom plate 610 and extends through an opening in top plate 606. Flexure elements 612 are connected to top and bottom plates 606 and 610, and are separated from post 622. Top plate 606 is not fixed to post 622 and therefore top plate 606 may rotate freely about post 622. Similar to the embodiment of FIG. 17A, device 600 comprises a housing or outer shell 616 and shell set screw holes 634 that are arranged circumferentially around the proximal rim of outer shell 616. In some embodiments, device 600 comprises top plate set screw holes 636 having threads arranged circumferentially on the peripheral rim of top plate or first joint member 606. In this example, the at least one flexure element is inserted into the cup of outer shell 616 and fixedly attached to the proximal rim of the cup. To prevent movement, the compliant mechanism is set in place using set screws positioned in shell set screw holes 634 and top plate set screw holes 636 to fix housing 616 to top plate 606.

In some embodiments, the at least one flexure element 612 are positioned radially and equidistantly about a central axis passing through the first and second joint members. In some embodiments, the first and second joint members may rotate in at least one degree of motion. In some embodiments, the first and second joint members may flex in the same plane.

Devices 300, 400, 500 and 600 describe implants wherein a compliant stem is formed that may be used for any orthopaedic implant that has a stem (e.g. knee, hip, shoulder, elbow, wrist, ankle, etc). The compliance allows for rotational motion without creating large shear stresses at the bone-implant interface.

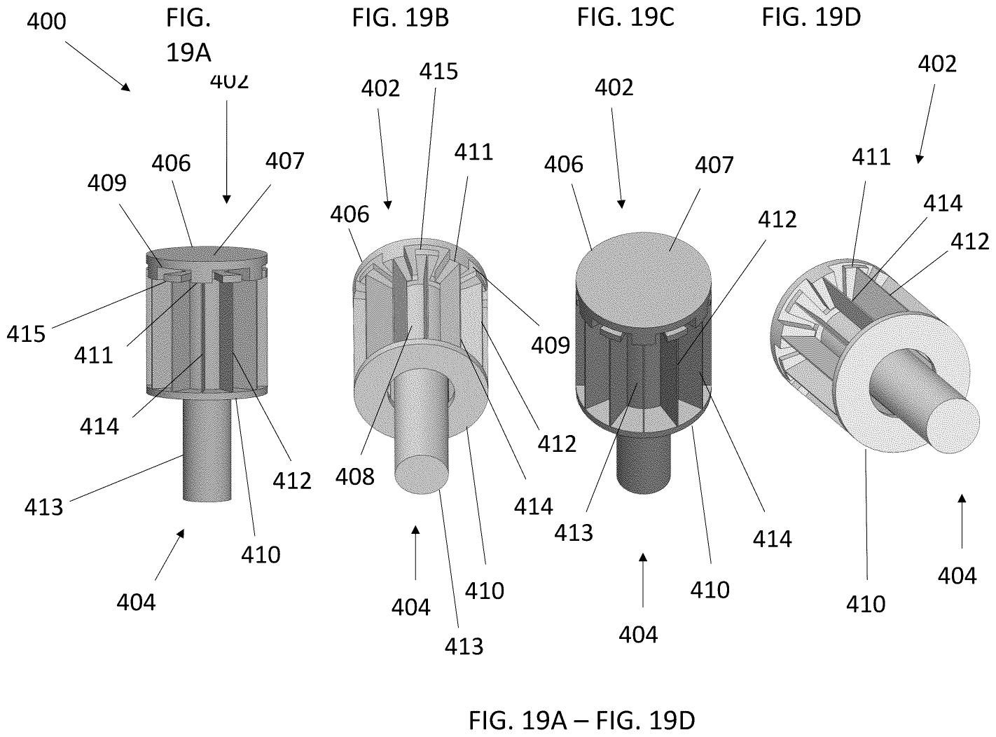

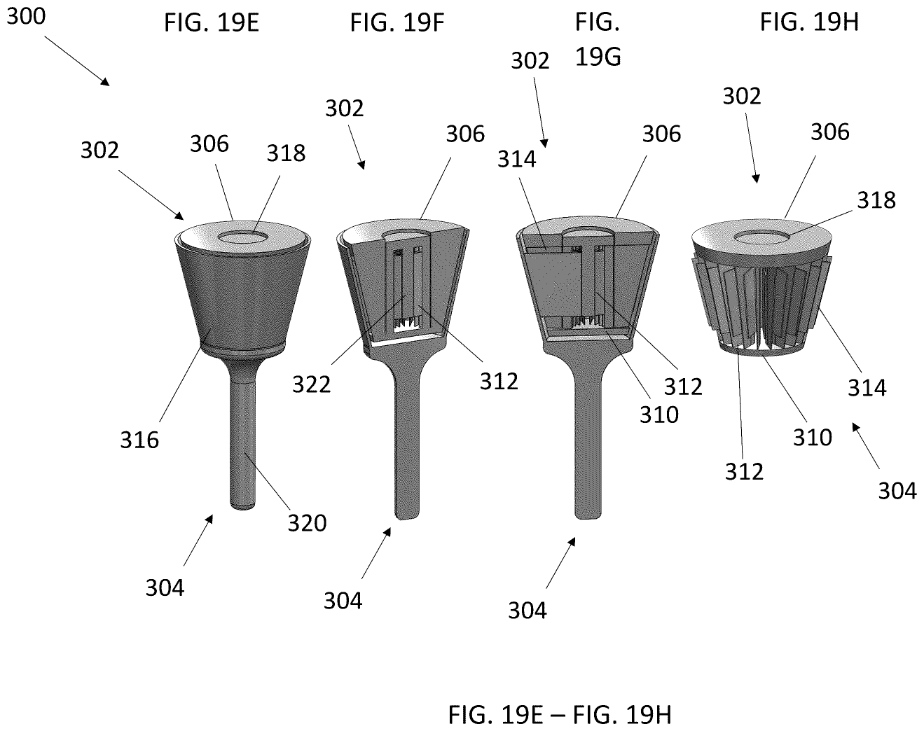

Referring now to FIG. 19A through FIG. 19D, another exemplary implant device 400 is shown. Device 400 comprises a proximal end 402, a distal end 404, a top plate 406, a central post, a bottom plate 410, a plurality of flexure elements 412, and a plurality of blades 414.

Top plate 406 comprises a top surface 407 and a bottom surface 409. Bottom surface 409 comprises a plurality of ridges 411. In some embodiments, plurality of ridges 411 may have any shape known to one skilled in the art. In some embodiments, plurality of ridges 411 have a height ranging between about 0.1 cm and about 10 cm. In some embodiments, plurality of ridges 411 have a width ranging between about 0.1 cm to about 3 cm. In some embodiments, the distance between two ridges 411 may range between about 0.1 cm to about 3 cm.

Top plate 406 is connected to a proximal anchor at top surface 407 and connected to plurality of blades 414 through plurality of ridges 411 at bottom surface 409. In some embodiments, top plate 406 may have any shapes known to one skilled in the art including but not limited to circular, oval, etc. In some embodiments, top plate 406 may have any diameter ranging between approximately 0.5 cm to about 5 cm. In some embodiments, the proximal anchor is configured to attach device 400 to surrounding bone via any method known to one skilled in the art including but not limited to intramedullary fixation, extramedullary fixation, surface osscointegration, etc.

Plurality of blades 414 are connected to bottom plate 410 at distal end 404. In some embodiments, plurality of blades 414 may have any shapes known to one skilled in the art including but not limited to rectangular, trapezoidal, etc.

In some embodiments, plurality of blades 414 may have a height ranging between about 0.3 cm to about 20 cm. In some embodiments, plurality of blades 414 may have a width ranging between about 0.1 cm to about 3 cm. In some embodiments, plurality of blades 414 may have a thickness of up to about 0.3 cm.

Central post comprises a post body 413 and a plurality of projections 415 extending laterally from proximal end 402 of post body 413. Plurality of projections 415 are connected to plurality of flexure elements 412 at proximal end 402. Plurality of projections 415 are positioned between two ridges 411, wherein the distance between two ridges 411 is configured to define the rotational degree of plurality of flexure element 412 in each direction. In some embodiments, plurality of flexure elements 412 may have a 1-90 degree Freedom of motion in each direction. In some embodiments, post body 413 may have any cross-sectional shape known to one skilled in the art including but not limited to circular. In some embodiments, post body 413 may have a width ranging between about 0.5 cm to about 5 cm. In some embodiments, post body 413 may have a height ranging between about 1 cm to about 30 cm.

Central post rotates relative to bottom plate 410, by bending of the flexure elements between the proximal end of central post and bottom plate 410. Bottom plate 410 simultaneously rotates relative to top plate 406, which would be anchored to bone, via bending of the flexure elements between top and bottom plate. It is to be noted that in device 300, all of the flexure elements are in tension when the device is in compression. In device 400, half of the flexure elements are in tension while the other half are in compression.

Plurality of flexure elements 412 are connected to plurality of ridges 412 at proximal end 402 and are connected to bottom plate 410 at distal end 404. In some embodiments, plurality of flexure elements 412 may be connected to post body 413. In some embodiments, flexure element 412 may have any shapes known to one skilled in the art including but not limited to: rectangular, trapezoidal, etc. In some embodiments, flexure elements 412 may have a height ranging between about 0.3 cm to about 20 cm. In some embodiments, plurality of flexure elements 412 may have a width ranging between about 0.1 cm to about 3 cm. In some embodiments, plurality of flexure elements 412 may have a thickness of up to about 0.3 cm. In some embodiments, the height of each flexure element 412 may be shorter than each blade 414. In some embodiments, the height of each flexure element 412 is similar to the height of each blade 414.

Plurality of flexure elements 412 and plurality of blades 414 are configured to provide rotational compliance as well as compressive compliance in proximal and distal directions.

In some embodiments, top plate 406 may have a larger diameter than bottom plate 410. In some embodiments, top plate 406 may have the same diameter as bottom plate 410. In some embodiments, top plate 406 may have a smaller diameter than bottom plate 410.

In some embodiments, device 400 may further comprise an outer shell to protect the surrounding tissue. In some embodiments, plurality of blades 414 may be connected to the outer shell. In some embodiments, the outer shell may be made from any material known to one skilled in the art including but not limited to pure metals, metal alloys, polymers, ceramics, metallic glasses, or combinations thereof. In some embodiments, the outer shell may be made from a biocompatible material. In some embodiments, the outer shell may be coated in a biocompatible material. In some embodiments, the outer shell may comprise Ti6-Al4v. In some embodiments, the outer shell may comprise Cr-Co. In some embodiments, the outer shell may comprise SS316.

In some embodiments, the outer shell may be connected to a distal anchor at distal end 404. In some embodiments, the distal anchor is configured to attach device 400 to surrounding bone via any method known to one skilled in the art including but not limited to intramedullary fixation, extramedullary fixation, surface osseointegration, bone cementation, etc.

Referring now to FIG. 19E through FIG. 19H, another exemplary implant device is shown. Device 300 comprises a proximal end 302, a distal end 304, a top plate 306, a central post, a bottom ring 310, a plurality of flexure elements 312, a plurality of blades 314, and an outer shell 316.

Top plate 306 comprises an opening 318 positioned in the center of top plate 306. In some embodiments, top plate 306 may have any shapes known to one skilled in the art including but not limited to circular, oval, etc. In some embodiments, top plate 306 may have any diameter ranging between approximately 0.5 cm to 5 cm. In some embodiments, opening 318 may have any shapes known to one skilled in the art including but not limited to circular, oval, etc. Top plate 306 is connected to a proximal anchor at proximal end 302 and connected to plurality of flexure elements 312 at distal end 304. In some embodiments, the proximal anchor is configured to attach device 300 to surrounding bone via any method known to one skilled in the art including but not limited to intramedullary fixation, extramedullary fixation, surface osseointegration, etc.

Central post is positioned inside opening 318 and comprises a top plate 306, a central shaft 322 and a plurality of flexure elements 312. Central shaft 322 is connected to top plate 306 at proximal end 302 and extends in a distal direction from top plate 306 towards bottom ring 310, but does not touch bottom ring 310. In some embodiments, central shaft 322 may have any cross-sectional shape known to one skilled in the art including but not limited to circular. In some embodiments, central shaft 322 may have a diameter ranging between about 0.2 cm and 4 cm. In some embodiments, central shaft 322 may have a height ranging between about 0.3 cm to about 20 cm.

Plurality of flexure elements 312 is connected to top plate 306 at proximal end 302 and is connected to bottom ring 310 at distal end 304. In some embodiments, plurality of flexure elements 312 may have any shapes known to one skilled in the art including but not limited to rectangular.

Plurality of flexure elements 312 are connected to top plate 306 at proximal end 302 and to bottom ring 310 at distal end 304. In some embodiments, plurality of flexure elements 312 may have any shapes known to one skilled in the art including but not limited to rectangular, trapezoidal, etc. In some embodiments, plurality of flexure elements 312 may have a height ranging between about 0.3 cm to about 20 cm.

In some embodiments, plurality of flexure elements 312 may have a width ranging between about 0.1 cm to about 3 cm. In some embodiments, plurality of flexure elements 312 may have a thickness of up to about 0.3 cm.

In some embodiments, top plate 306 may have a larger diameter than bottom ring 310. In some embodiments, top plate 306 may have the same diameter as bottom ring 310. In some embodiments, top plate 306 may have a smaller diameter than bottom ring 310

In some embodiments, a plurality of flexure elements 314 may have any shapes known to one skilled in the art including but not limited to rectangular, trapezoidal, etc. In some embodiments, plurality of flexure elements 314 may have a height ranging between about 0.3 cm and about 20 cm. In some embodiments, plurality of flexure elements 314 may have a width ranging between about 0.1 cm to about 3 cm. In some embodiments, plurality of flexure elements 314 may have a thickness of up to about 0.3 cm. In some embodiments, the height of each blade 314 may be shorter than each flexure element 312.

Plurality of blades 314 are positioned between two flexure element 312 and are configured to limit the rotational degree of plurality of flexure element 312 in each direction. In some embodiments, plurality of flexure elements 312 may have a 1-90 degree Freedom of motion in each direction.

Plurality of flexure elements 312 and plurality of blades 314 are configured to provide rotational compliance as well as compressive compliance in proximal and distal directions.

Outer shell 316 is configured to protect the surrounding tissue and at least partially enclose plurality of flexure element 312. In some embodiments, outer shell 316 may be made from any material known to one skilled in the art including but not limited to pure metals, metal alloys, polymers, ceramics, metallic glasses, or combinations thereof. In some embodiments, outer shell 316 may be made from a biocompatible material. In some embodiments, outer shell 316 may be coated in a biocompatible material. In some embodiments, outer shell 316 may comprise Ti6-A14v. In some embodiments, outer shell 316 may comprise Cr-Co. In some embodiments, outer shell 316 may comprise SS316.

Outer shell 316 is connected to a distal anchor 320 at distal end 304. Distal anchor 320 is configured to attach device 300 to surrounding bone via any method known to one skilled in the art including but not limited to intramedullary fixation, extramedullary fixation, surface osseointegration, etc.

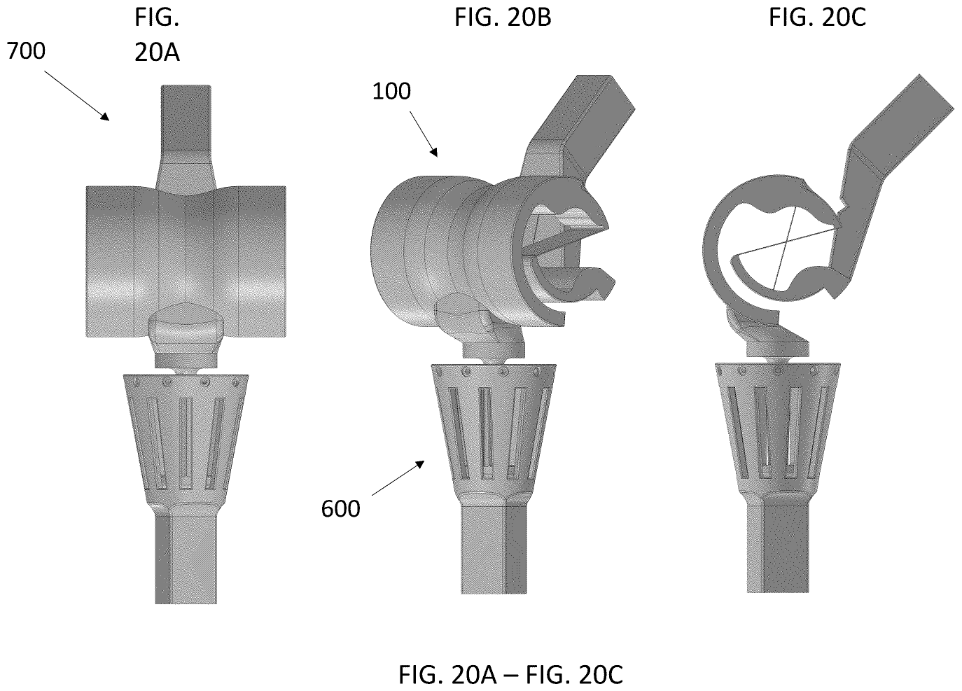

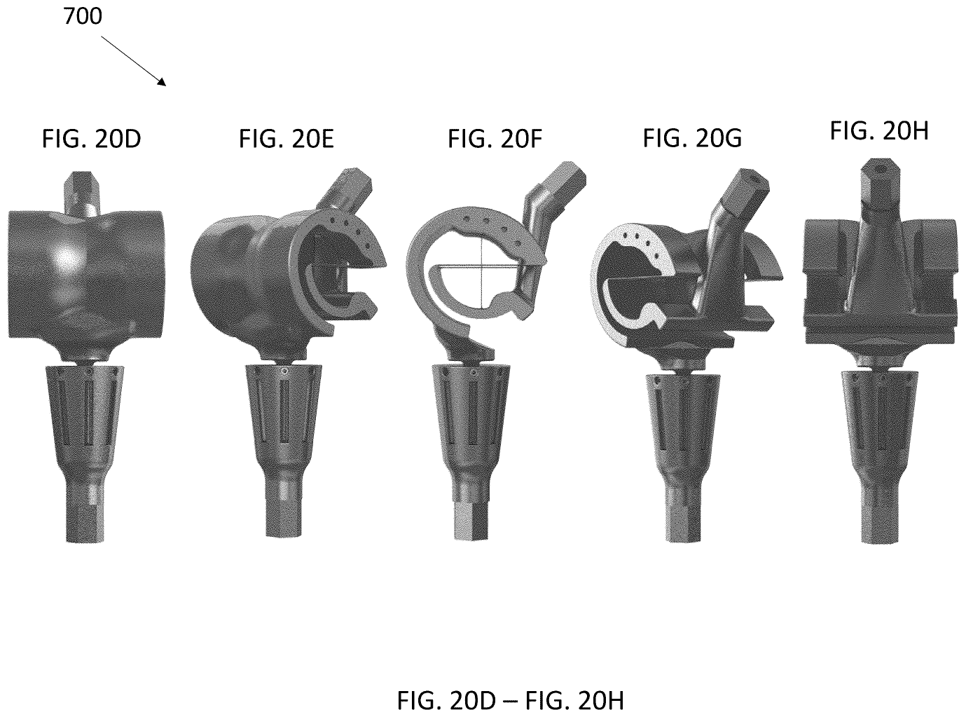

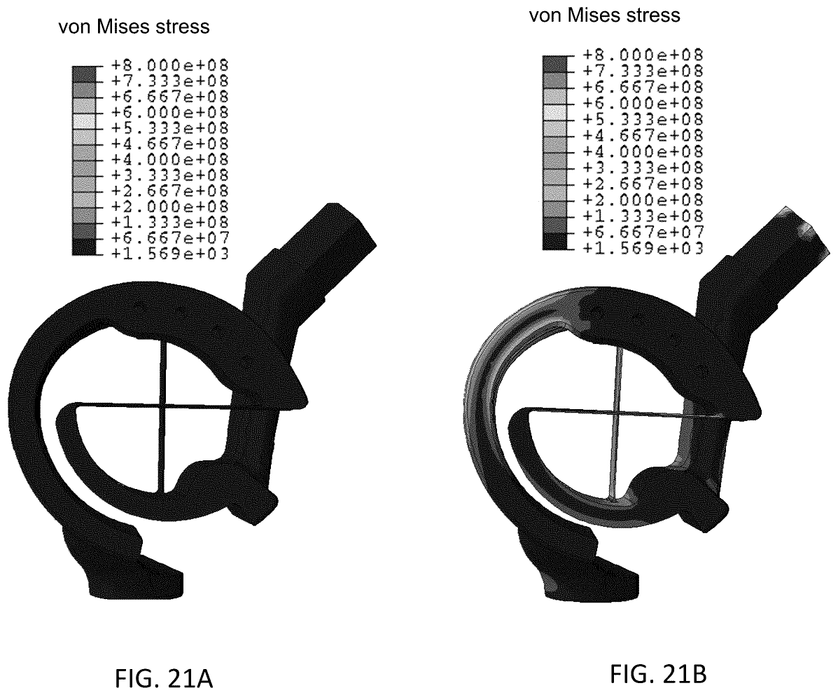

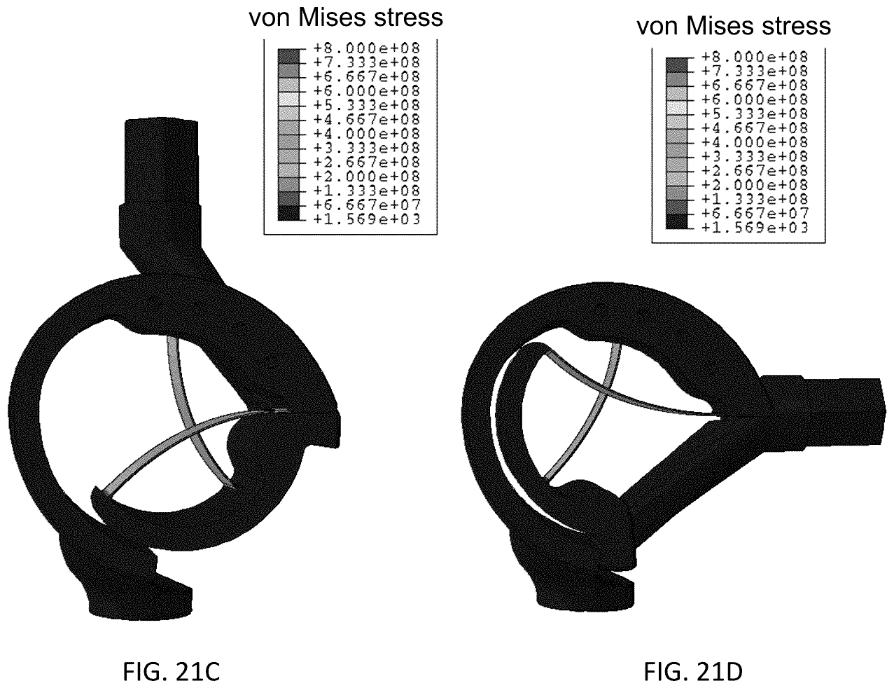

Referring now to FIG. 20A through FIG. 20H shown is an exemplary 2-degree of freedom implant device 700 of the present invention. In some embodiments, at least one implant device of the present invention is attached in series to allow at least one degree of freedom in movement. In an example, a first implant device allows a first degree of freedom in movement, and a second implant device allows a second degree of freedom in movement. In the example of FIG. 20A though FIG. 20H, an implant device 100 of the present invention is fixedly attached in series to an implant device 600 of the present invention to allow two degrees of freedom.

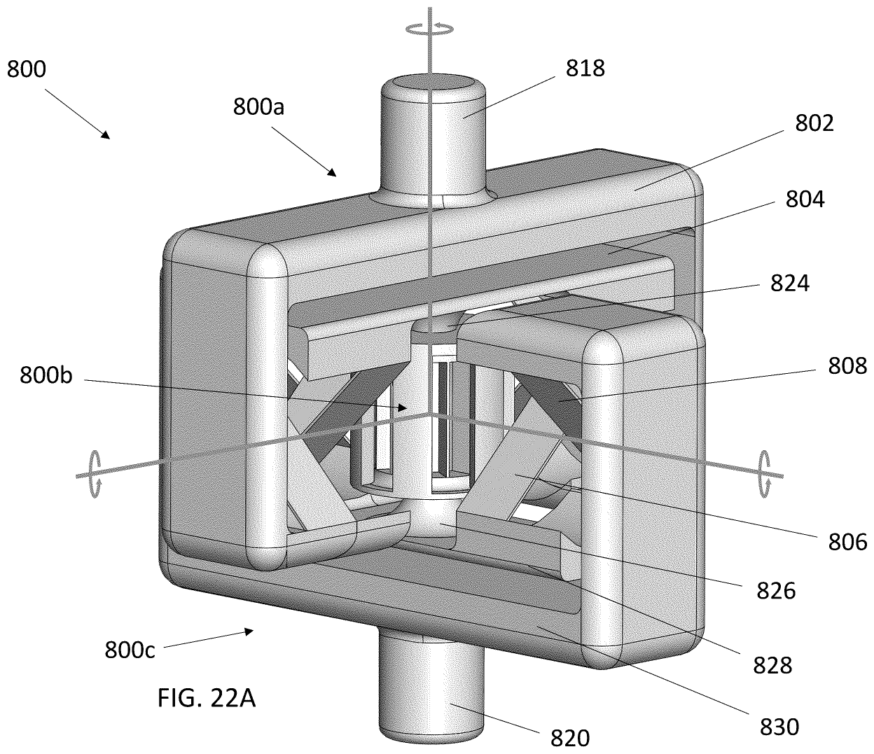

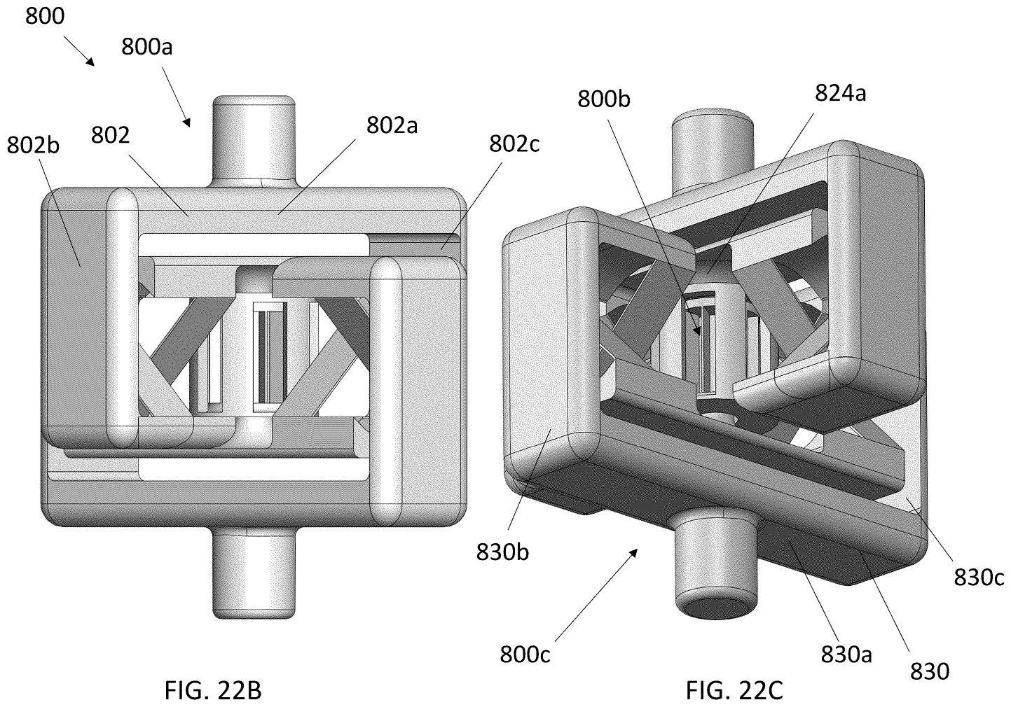

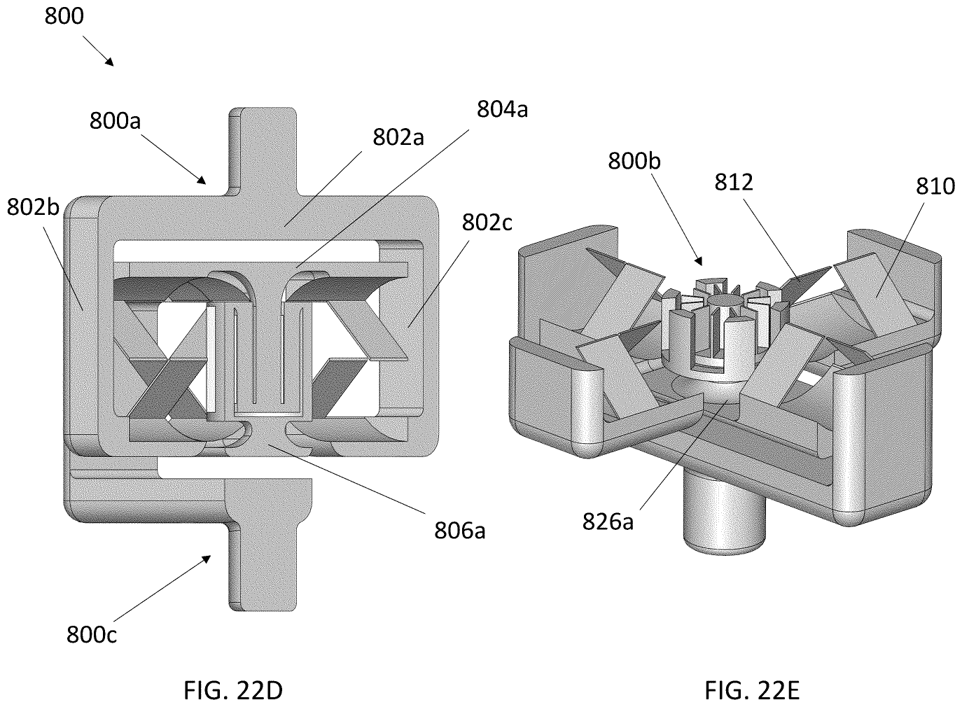

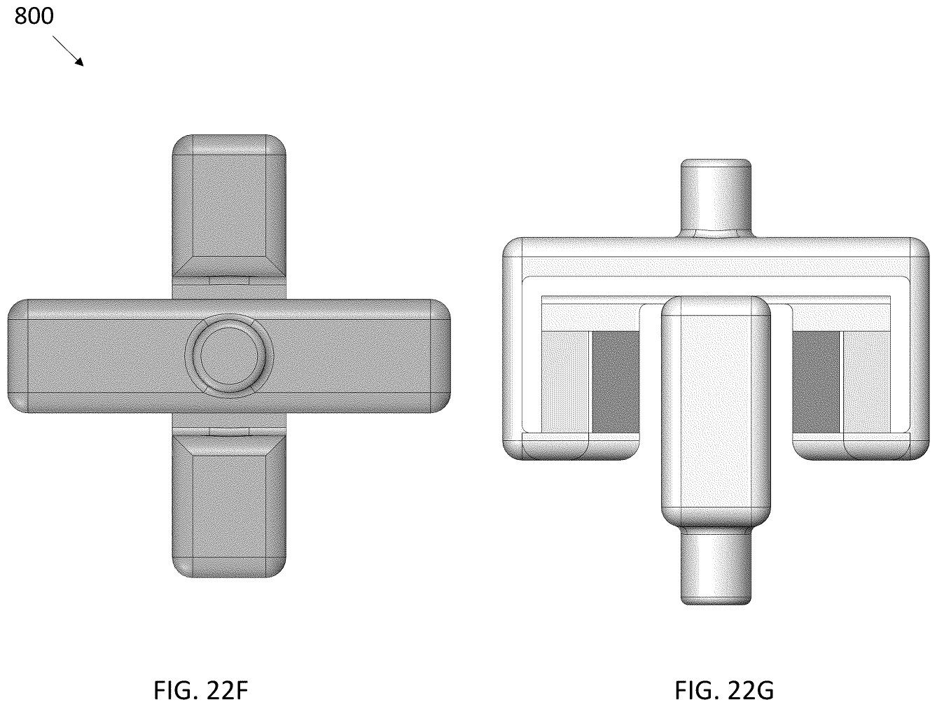

Referring now to FIG. 22A through FIG. 22G, shown is an exemplary 3-degree of freedom implant device 800 of the present invention. In some embodiments, at least one implant device of the present invention is attached in series to allow at least one degree of freedom in movement. In this example, a first implant device allows a first degree of freedom in movement, a second implant device allows a second degree of freedom in movement, and a third implant device allows a third degree of freedom in movement. In some embodiments, device 800 comprises a first compliant device 800a, a second compliant device 800b, and a third compliant device 800c.

In some embodiments, device 800 comprises a first joint member 802, a second joint member 804, a third joint member 824, a fourth joint member 826, a fifth joint member 828, a sixth joint member 830 at least one flexure element (e.g. first flexure element 806 and second flexure element 808), a proximal anchor 818 and a distal anchor 820. In some embodiments, first joint member 802, second joint member 804, third joint member 824, fourth joint member 826, fifth joint member 828, and sixth joint member 830 may have any shape known to one skilled in the art.

Now referring to FIG. 22B, in some embodiments, first compliant device 800a comprises a first joint member 802 having a bridge member 802a, with a first arm 802b and a second arm 802c extending from either end of the bridge member. In some embodiments, third compliant device 800c comprises a sixth joint member 830 having a bridge member 830a, with a first arm 830b and a second arm 830c extending from either end of the bridge member. The first arm and second arm extending from either end of each bridge member are connection points for at least one flexure element. In some embodiments, second compliant device 800b comprises third joint member 824 and fourth joint member 826, each joint member having a bridge member (824A and 826A, respectively). In some embodiments, the first joint member 802 has a top end region for the connecting to proximal anchor 818, and a bottom end region for connecting to at least one flexure element. In some embodiments, the sixth joint member 830 has a bottom end region for connecting to distal anchor 820, and a top end region for connecting to at least one flexure element.

In some embodiments, the at least one flexure element may have any number of flexure elements applicable and known to one skilled in the art. In some embodiments, the at least one flexible structure comprises a first flexure element 806, a second flexure element 806.

In some embodiments, the implant device comprises a third joint member 824 having an inner surface and an outer surface, a fourth joint member 826 having an inner surface and an outer surface, a fifth joint member 828 having an inner surface and an outer surface, a sixth joint member 830 having an inner surface and an outer surface, at least one flexure element having first and second ends, wherein the first end is connected to the inner surface of third joint member 824 and the second end is connected to the inner surface of fourth joint member 826, at least one flexure element having first and second ends, wherein the first end is connected to the inner surface of fifth joint member 828 and the second end is connected to the inner surface of sixth joint member 830, wherein the inner surface of second joint member 804 is connected to the outer surface of third joint member 824, wherein the outer surface of fourth joint member 826 is connected to the inner surface of fifth joint member 826, wherein third joint member 824 and fourth joint member 826 are configured to move relative to each other within a second degree of freedom that is different than the first degree of freedom of movement between first joint member 802 and second joint member 804, and wherein fifth joint member 828 and sixth joint member 830 are configured to move relative to each other within a third degree of freedom that is different than the second degree of freedom of movement between third joint member 824 and fourth joint member 826.

In some embodiments, the first degree of freedom is translational, the second degree of freedom is rotational, and the third degree of freedom is translational. In some embodiments, the first degree of freedom is rotational, the second degree of freedom is translational, and the third degree of freedom is rotational. In some embodiments, the first, second and third degrees of freedom are each translational. In some embodiments, the first, second and third degrees of freedom are each rotational. In some embodiments, the device further comprises a first tissue anchor (proximal anchor 818) extending from the outer surface of first joint member 802, and a second tissue anchor (distal anchor 820) extending from the outer surface of sixth joint member 830. In some embodiments, the first and second tissue anchors are each configured to engage bone via at least one selected from the group consisting of intramedullary fixation, extramedullary fixation, osseointegration and combinations thereof.

First joint member 802 and fourth joint member 808 have the same structure in a flipped and perpendicular orientation, with second joint member 804 and third joint member 806 being disposed between first joint member 802 and fourth joint member 808.

In some embodiments, first flexure element 810 and second flexure element 812 may function as springs and intersect independent of one another to provide at least one axis of rotation for joint member 802, second joint member 804, third joint member 806, and fourth joint member 808.

In some embodiments, first flexure element 810, second flexure element 812 may intersect at any point along their length. In some embodiments, the center of rotation may be approximated by the intersection point, but moves as the flexure elements deform. In some embodiments, flexure elements may have different lengths and can be oriented at any angle, wherein changing any of these properties affects the mechanical behavior of the mechanism. In some embodiments, flexure elements may be oriented such that the first flexure element 810 and second flexure element 812 form a crossing pattern. In some embodiments, flexure elements may be oriented such that the first flexure element 910 and second flexure element 812 are substantially parallel.

Proximal anchor 818 is attached to first joint member 802 at one end and is configured to attach device 800 to bone at the opposing end. Distal anchor 820 is attached to fourth joint member 808 at one end and is configured to attach device 800 to bone at the opposing end.

In some embodiments, device 800 may be anchored to the surrounding bones through proximal anchor 818 and distal anchor 820 via any method known to one skilled in the art including but not limited to intramedullary fixation, extramedullary fixation, surface osseointegration, etc. In some embodiments, device 800 may be anchored to surrounding bone through proximal anchor 818 and distal anchor 820 by intramedullary fixation. In some embodiments, any of a number of strategies for intramedullary fixation may be used to anchor device to the surrounding bone including but not limited to: long stem, short stem, threaded intramedullary screws, press-fit or cemented stems, nails, tapped and threaded implants, compression-style implants, porous coated for bone ingrowth, etc. In some embodiments, intraosseous rods or pins may be used for fixation to cancellous bone.

In some embodiments, device 800 may be anchored to surrounding bone through proximal anchor 818 and distal anchor 820 by extramedullary fixation. In some embodiments, any of a number of strategies for intramedullary fixation may be used to anchor device to the surrounding bone including but not limited to: plates and screws. In some embodiments, conventional orthopedic hardware may be used for extra-osseous fixation including but not limited to customized plates and screws, standardized plates and screws, etc. In some embodiments, device 800 may be anchored to surrounding bone by ingrowth on a porous surface. In some embodiments, bony surfaces may be prepared intraoperatively for ingrowth onto a porous surface (e.g., porous coating for bone ingrowth as in hip replacement acetabular cups). In some embodiments, initial stability may be aided by combination with any other fixation strategy.

In some embodiments, proximal anchor 818 and distal anchor 820 may have any shapes/geometries known to one skilled in the art including but not limited to cylindrical, cubical, etc.

In some embodiments, proximal anchor 818 and distal anchor 820 may be made from any material including but not limited to titanium, Ti6-Al4v, Cr-Co, SS316. In some embodiments, proximal anchor 818 and distal anchor 820 may be porous. In some embodiments, proximal anchor 818 and distal anchor 820 may be further coated with any material known to one skilled in the art including but not limited to pure metals, metal alloys, polymers, ceramics, metallic glasses, or combinations thereof. In some embodiments, proximal anchor 818 and distal anchor 820 may comprise titanium. In some embodiments, proximal anchor 818 and distal anchor 820 may comprise a biocompatible material.

In some embodiments, device 800 may be used for common orthopedic procedures including but not limited to total hip replacement, total knee replacement, total shoulder replacement etc. In some embodiments, it is possible to surgically alter the joint (e.g., foot) to facilitate fixation. In one exemplary embodiment, joints may be fused to eliminate motion that would be deleterious to bony ingrowth and osseointegration. In some embodiments, bones may be fused together to create larger bony surfaces to which the implant could be fixed. In some embodiments, bones may be removed or relocated to create more space for the implant.

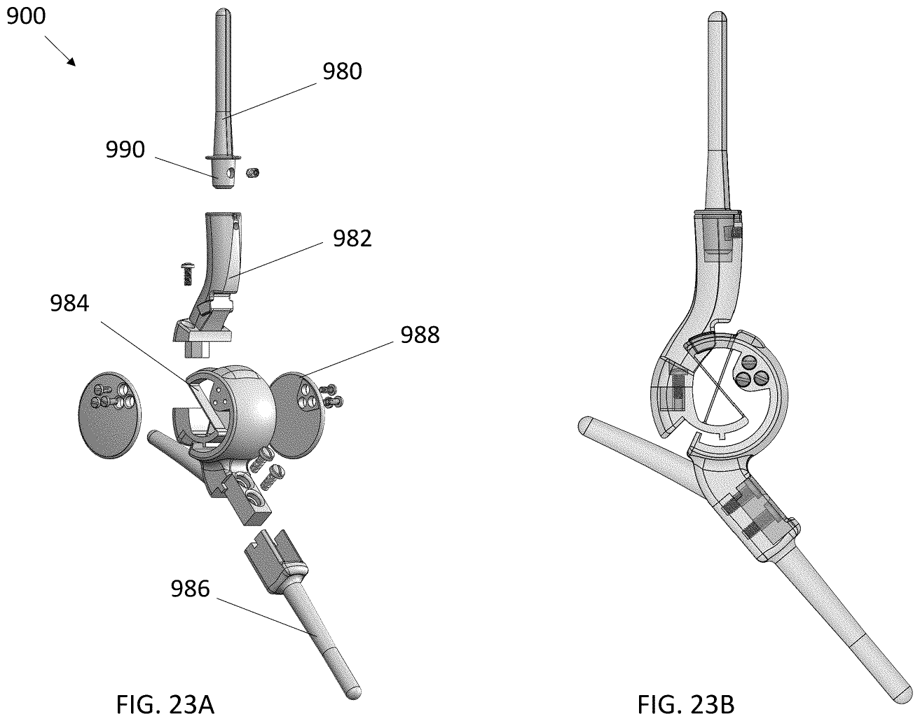

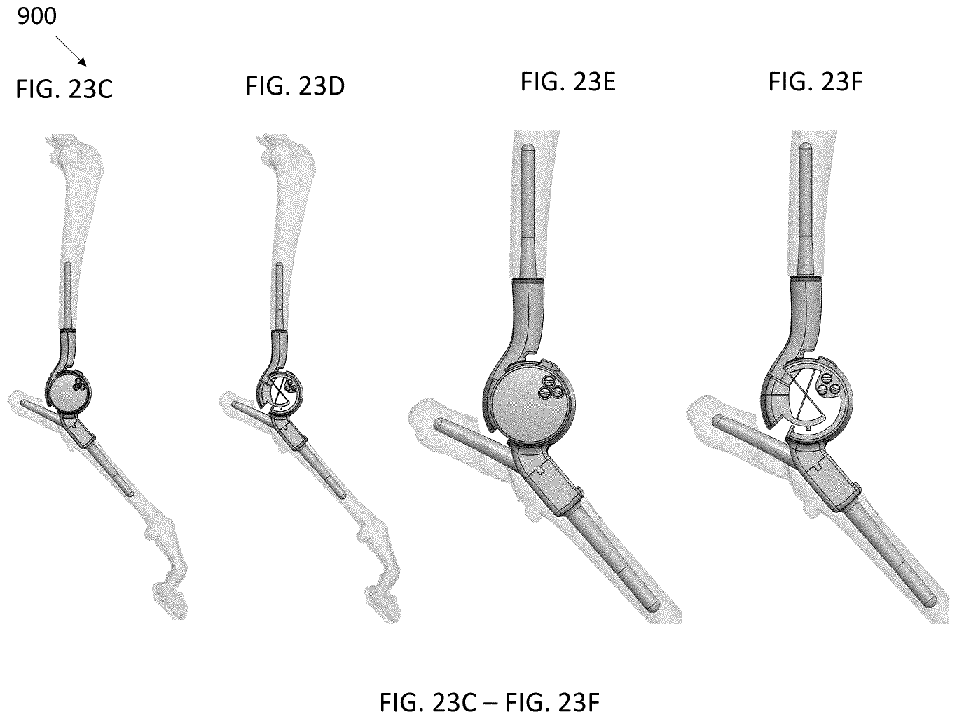

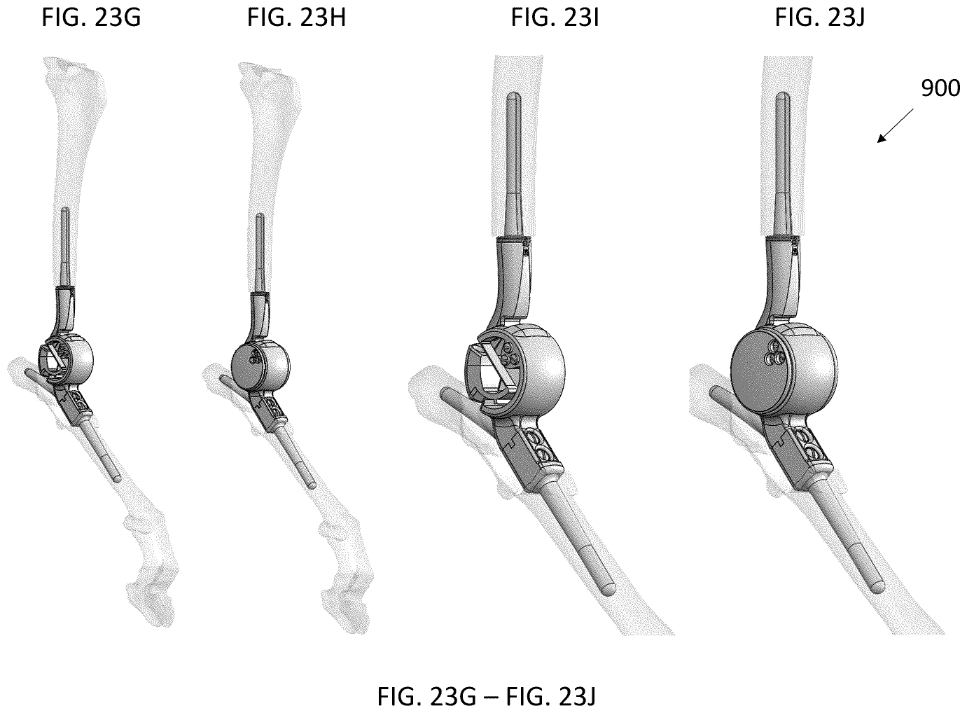

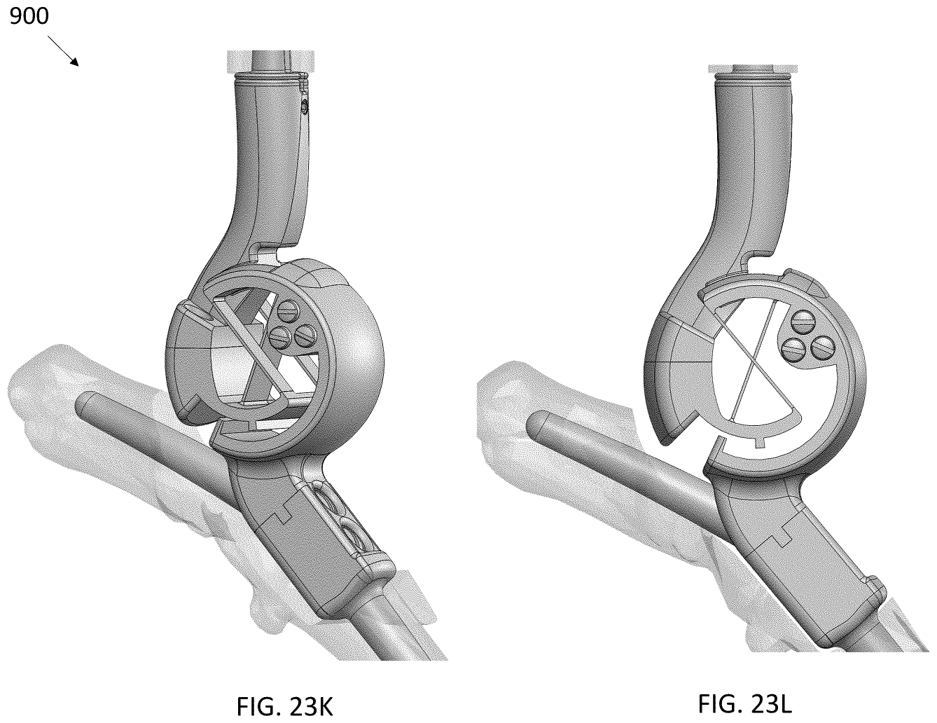

FIG. 23A through FIG. 23J depicts an exemplary implant device 900 of the present invention for an animal model. In some embodiments, device 900 comprises tibial stem 980, stem adapter 982, flexure element 984, metatarsal stem 986, implant cover 988, and morse taper 990. Tibial stem 980 and metatarsal stem 986 are fixedly attached to flexure element 984 by any method known to one skilled in the art.

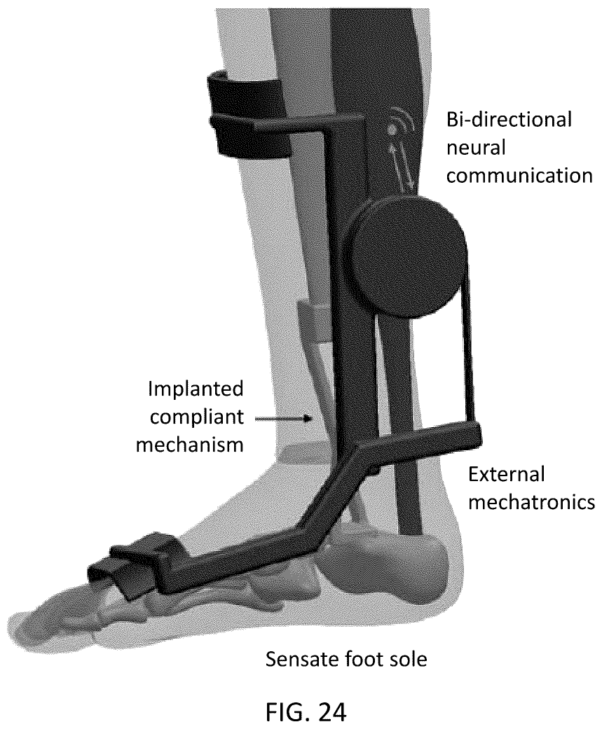

In some embodiments, device 100 through device 900 may be implemented in parallel with an external exoskeleton, to provide power or additional passive assistance to the subject (FIG. 24). In some embodiments, an external exoskeleton may be unidirectionally interfaced with the subject's nervous system. In some embodiments, an external exoskeleton may be bi-directionally interfaced with the subject's nervous system. In some embodiments, this configuration comprises a fully sensational biological limb. In some embodiments, this configuration comprises a neurally-controlled mechanical joint. In some embodiments, this configuration comprises a closed skin envelope that is robust to infection. In some embodiments, this configuration comprises external mechatronics that are easily accessible for repair or upgrade.

In some embodiments, device 100 through device 900 may be used in parallel with a separate spring/compliant mechanism including but not limited to coil spring, second compliant mechanism, etc. to increase the energy storage capacity of the joint. In some embodiments, this could be a fixed component that is integrated into the compliant mechanism, or a modular component that can be exchanged depending on patient anatomy or specifics of the pathology.

In some embodiments, device 100 through device 900 may be used in rehabilitative applications. In some embodiments, device 100 through device 900 may be used in preventive/augmentative applications. In some embodiments, device 100 through device 900 may be used for treating a subject having a diagnosed pathology. In some embodiments, device 100 through device 900 may be used for a subject seeking prophylactic injury prevention. In some embodiments, device 100 through device 900 may be used for a subject to enhance movement ability beyond their baseline capacity. In some embodiments, device 100 through device 900 may be used in parallel with existing joints, to manipulate their mechanical properties and provide energy storage and return. In one exemplary embodiment, device 100 through device 900 may be implanted in parallel with the joints of a subject with joint weakness, to stiffen the joint and enable improved function.

In some embodiments, any fabrication method including but not limited to traditional subtractive manufacturing with no assembly, traditional subtractive manufacturing with assembly, additive manufacturing, and combinations thereof may be used to fabricate device 100 through device 900 . In some embodiments, a traditional subtractive manufacturing may be used. In some embodiments, device 100 through device 900 may be manufactured as a single piece using the traditional subtractive manufacturing method. In some embodiments, both devices may be manufactured using traditional subtractive manufacturing with no assembly. In some embodiments, both devices may be manufactured using traditional subtractive manufacturing with assembly.

In some embodiments, both devices may be manufactured with at least two distinct pieces. In some embodiments, the at least two distinct pieces may be assembled prior to sterilization and implantation. In some embodiments, the joints between at least two distinct pieces may be fixed using any method known to one skilled in the art including but not limited to welding, bolting, adhesives, interference fits, heat modulated interference fits, etc. In some embodiments, each individual component of the at least two distinct pieces may be assembled at a low temperature, so that the pieces expand to interference fits when exposed to the body's heat. In some embodiments, device may be manufactured using additive manufacturing techniques including but not limited to 3D printing. In some embodiments, both devices may be 3D printed as a single piece. In some embodiments, both devices both devices may be 3D printed as at least two pieces that can be assembled.

In some embodiments, a potential option for manufacturing is to precision machine the flexure blades as shims from bulk material. These shim blades could then be inserted into a housing that is conventionally machined, 3d printed, injection molded, or investment casted. The shims could be attached to the housing via a press fit, thermal expansion fit, weld, bolt, or any combination of these options.

Alternatively, in another embodiment, the shim blades could be inset into a mold of the implant to be used for investment casting of the housing. This would allow for a bulk weld between the shim blades and the housing, and would eliminate the need for the molten material to flow into the small blade profiles. All or part of the shim blades could be actively or passively cooled to keep the blades from being affected by the molten housing material.

### Methods of the Invention

The present invention provides a method for facilitating the design of orthopedic implants having a compliant mechanism for limb restoration. In some embodiments, the method of present invention is configured to integrate biomechanical, analytical, and finite element modeling to produce-from a set of predefined target biomechanics-optimized geometry and placement of flexure elements within a compliant mechanism. In some embodiments, the method of the present invention may be used to design compliant implantable prostheses for all of the body's joints. In some embodiments, the method of the present invention may be used to correct diverse pathologies including but not limited to arthritis, trauma, tumor, congenital deformity, infection, diabetic arthropathy, etc. In some embodiments, the method of the present invention may result in a joint-and-pathology-specific compliant implant design that is optimized to restore limb biomechanics.

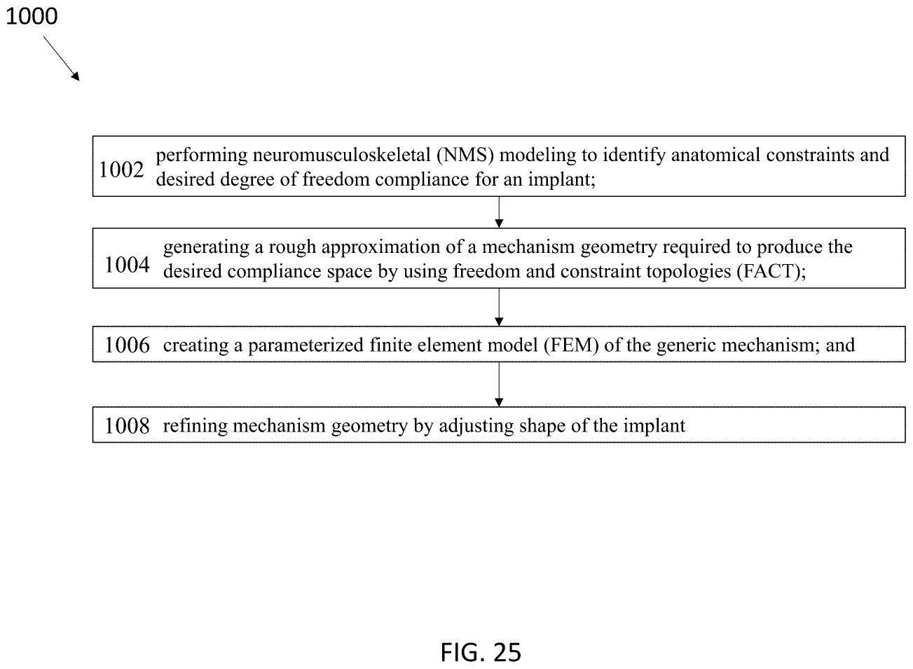

Referring now to FIG. 25, an exemplary method 1000 of designing compliant implantable prostheses is depicted. Method 1000 begins with step 1002, wherein neuromusculoskeletal (NMS) modeling or any biomechanical modeling is performed on a subject to identify anatomical constraints and desired degree of freedom compliance for an implant.

In step 1004, freedom, and constraint topologies (FACT) is used to generate a rough approximation of a mechanism geometry required to produce the desired compliance space. In some embodiments, FACT may be used to codify general compliant systems of any geometry. In some embodiments, FACT links infinitesimally small motions including but not limited to degrees of freedom, or “DOFs” to the complete design space of compliant solutions that achieve those motions. In some embodiments FACT is configured to open the door for rapid identification of application-specific compliant geometries. In some embodiments, FACT may be paired with recent computational advances to allow optimization of these geometries. In some embodiments, FACT may be configured to enable the design of compliant mechanisms with specified mechanical behaviors.

In step 1006, a parameterized finite element model (FEM) of the generic mechanism is generated from a quasi-static mechanic's model of the implant under loads representative of those encountered during gait. In some embodiments, the model is built based on mechanism geometry and materials. In some embodiments, the model outputs deformations, stresses, stiffnesses, etc. in response to simulated applied loads.

In step 1008, mechanism geometry is refined by adjusting shape of the implant. In some embodiments, shape of the implant may be adjusted based on its mechanics and fatigue life.

In some aspects, the present invention relates to a method of designing compliant implantable prostheses comprising the steps of, performing neuromusculoskeletal (NMS) modeling on a subject to identify anatomical constraints and desired degree of freedom compliance for an implant, generating a rough approximation of a mechanism geometry required to produce the desired compliance space by using freedom and constraint topologies (FACT), creating a parameterized finite element model (FEM) of the generic mechanism; from a quasi-static mechanic's model of the implant under loads representative of those encountered during gait, and refining mechanism geometry by adjusting shape of the implant.

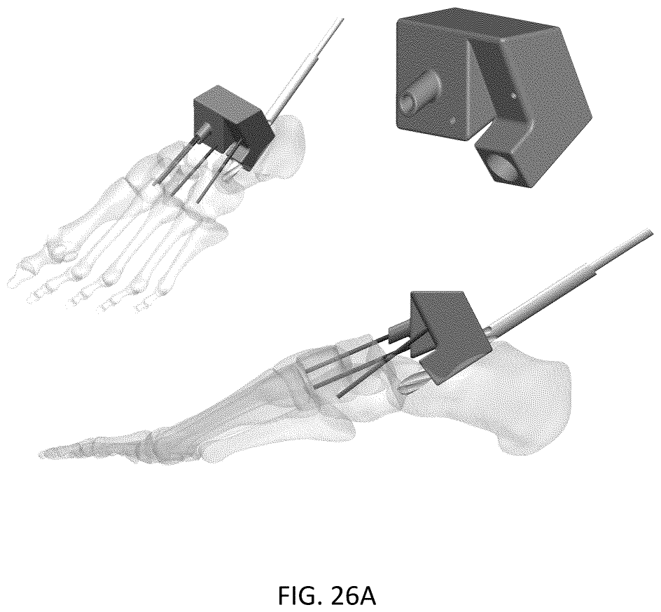

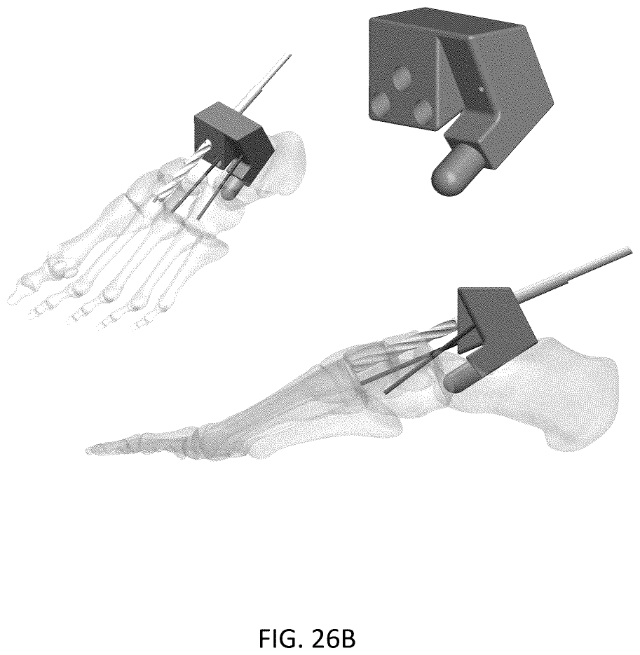

In some aspects, the present invention relates to a drill guide for the implantation of at least one implant device of the present invention. Now referring to FIG. 26A and FIG. 26B, shown is an exemplary drill guide for the implantation of at least one implant device of the present invention. Shown in FIG. 26A is a calcaneal drill guide. Shown in FIG. 26B is a midfoot drill guide. Note the blue and red lines represent k-wires that are put in place to hold the guide in place and guide the drill.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples, therefore, specifically point out exemplary embodiments of the present invention, and are not to be construed as limiting in any way the remainder of the disclosure.

### Example 1

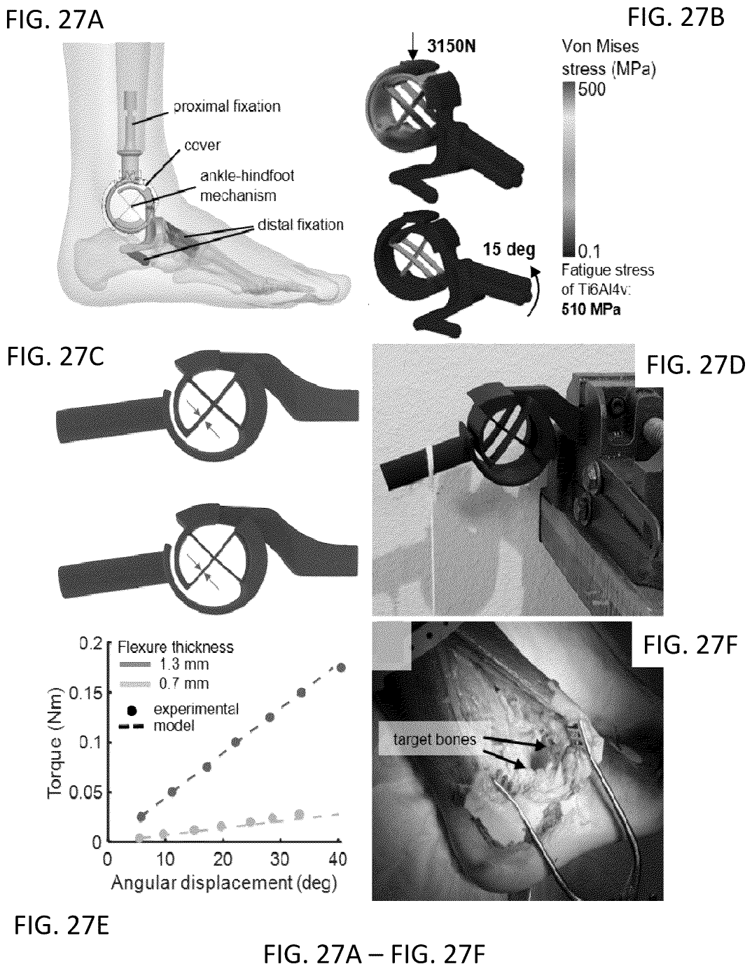

A preliminary modeling and experimental work was conducted to show feasibility of the compliant implant design for treatment of severe ankle and hindfoot pathology. This pilot work establishes that i) an implanted compliant mechanism of the appropriate size and constraint space for the ankle can withstand cyclic gait loads, ii) the behaviors of these mechanisms can be predictably tuned with small changes to flexure geometry, and iii) there is adequate bone for robust fixation in the target site. The generic ankle mechanism design (FIG. 27A) has high compliance about one axis, and tunable compliance in off-axis rotation and vertical compression. The design also includes a cover for the mechanism, which is important in preventing impingement and dead space within the artificial joint space. A preliminary finite element model (FEM) shows that the mechanism design in titanium (Ti6-A14v) can support ISO standard loads for ankle replacement, while remaining below the material's fatigue limit at 108 cycles (FIG. 27B); this corresponds to a conservative 5,000 steps per day for 91 years before failing from fatigue. Two prototype mechanisms of identical size but distinct flexure geometry (FIG. 27C) were also 3D-printed, and demonstrated that a FEM can accurately predict their radically different stiffnesses (FIG. 27D, FIG. 27E).

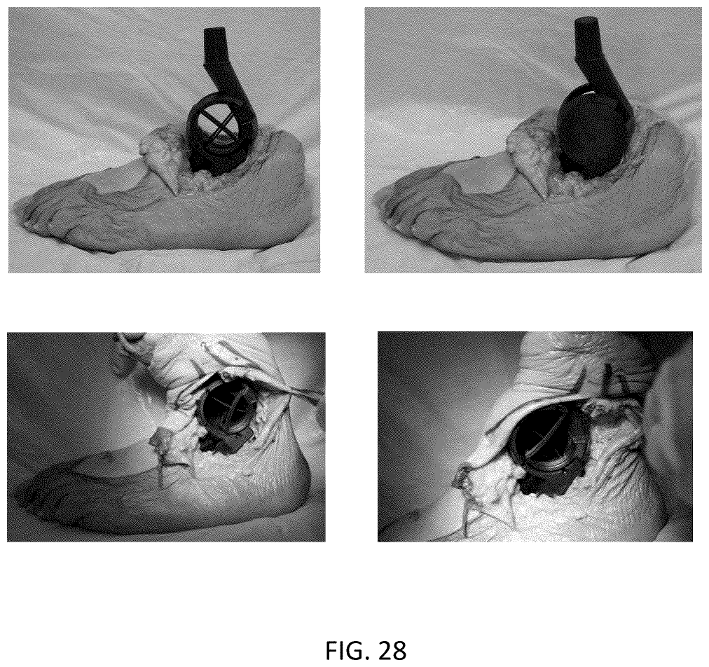

It is intended to remove the damaged talus, and anchor the ankle-hindfoot implant's distal end directly to the calcaneus, navicular, and cuneiform bones; although these bones are often used in fusion or other reconstructive approaches, the specific fixation approach requires development of new hardware. Toward this end, several dissections have been performed to assess these bones as a potential fixation site (FIG. 27F and FIG. 28) and to design a fixation strategy that leverages porous titanium to promote osseointegration with the target bones.

The disclosures of each and every patent, patent application, and publication cited herein are hereby each incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. An orthopedic device comprising:
a first joint member having an inner surface and an outer surface;
a second joint member having an inner surface and an outer surface; and
at least one flexure element having first and second ends, wherein the first end is connected to the inner surface of the first joint member and the second end is connected to the inner surface of the second joint member;
wherein the first and second joint members are configured to move relative to each other within a first degree of freedom.

2. The orthopedic device of claim 1, wherein the at least one flexure element comprises at least a first flexure element and a second flexure element.

3. The orthopedic device of claim 1, wherein:
the at least one flexure element comprises at least a first flexure element, a second flexure element and a third flexure element;
the first joint member comprises a top region, a bottom region and a central region between the top and bottom region along its length;
the second joint member comprises a top region, a bottom region and a central region between the top and bottom region along its length;
the first flexure element is connected at its first end to the inner surface of the bottom region of the first joint member and is connected at its second end to the inner surface of the top region of the second joint member;
the second flexure element is connected at its first end to the inner surface of the central region of the first joint member and is connected at its second end to the inner surface of the central region of the second joint member; and
the third flexure element is connected at its first end to the inner surface of the bottom region of the first joint member and is connected at its second end to the inner surface of the top region of the second joint member.

4. The orthopedic device of claim 1, wherein the at least one flexure element comprises a plurality of flexure elements.

5. The orthopedic device of claim 4, wherein the plurality of flexure elements are each oriented at the same angle relative to the inner surfaces of the first and second joint members, respectively.

6. The orthopedic device of claim 4, wherein the plurality of flexure elements are positioned radially and equidistantly about a central axis passing through the first and second joint members.

7. The orthopedic device of claim 6, further comprising:
an opening in the first joint member, wherein the opening has at least one sidewall; and
a central post having a first end fixed to or contiguous with the inner surface of the second joint member, and a second end extending through the opening of the first joint member such that the first joint member is rotatable around the post.

8. The orthopedic device of claim 6, further comprising a housing surrounding at least the first joint member, the second joint member and the plurality of flexure elements, wherein the first joint member is fixed to the housing.

9. The orthopedic device of claim 7, further comprising a first tissue anchor connected to the first joint member or the central post, and a second tissue anchor connected to the second joint member or the housing.

10. The orthopedic device of claim 8, further comprising a first tissue anchor connected to the second end of the central post, and a second tissue anchor connected to the housing.

11. The orthopedic device of claim 8, wherein:
the housing further comprises at least one recess; and
the second joint member further comprises at least one tab extending from the outer surface of the second joint member and positioned within the at least one recess of the housing;
wherein the at least one recess is sized to permit range-limiting movement of the at least one tab in the at least one recess when the second joint member is rotated relative to the first joint member.

12. The orthopedic device of claim 4, wherein the plurality of flexure elements are configured to deform when the first joint member moves relative to the second joint member within the first degree of freedom.

13. The orthopedic device of claim 4, wherein the first degree of freedom is rotational.

14. The orthopedic device of claim 1, further comprising at least one range-limiting element configured to restrict movement of the first joint member relative to the second joint member within the first degree of freedom.

15. The orthopedic device of claim 1, further comprising at least one range-limiting element configured to restrict movement of the second joint member relative to the first joint member within the first degree of freedom.

16. The orthopedic device of claim 1, further comprising:
a third joint member having an inner surface and an outer surface;
a fourth joint member having an inner surface and an outer surface;
at least one flexure element having first and second ends, wherein the first end is connected to the inner surface of the third joint member and the second end is connected to the inner surface of the fourth joint member;
wherein the third and fourth joint members are configured to move relative to each other within a second degree of freedom that is different than the first degree of freedom of movement between the first and second joint members; and
wherein the outer surface of the third joint member is connected to the outer surface of the second joint member.

17. The orthopedic device of claim 16, wherein the first and second degrees of freedom are each translational.

18. The orthopedic device of claim 16, wherein the first degree of freedom is translational and the second degree of freedom is rotational.

19. The orthopedic device of claim 1, further comprising:
a third joint member having an inner surface and an outer surface;
a fourth joint member having an inner surface and an outer surface;
a fifth joint member having an inner surface and an outer surface;
a sixth joint member having an inner surface and an outer surface;
at least one flexure element having first and second ends, wherein the first end is connected to the inner surface of the third joint member and the second end is connected to the inner surface of the fourth joint member;
at least one flexure element having first and second ends, wherein the first end is connected to the inner surface of the fifth joint member and the second end is connected to the inner surface of the sixth joint member;
wherein the inner surface of the second joint member is connected to the outer surface of the third joint member;
wherein the outer surface of the fourth joint member is connected to the inner surface of the fifth joint member;
wherein the third and fourth joint members are configured to move relative to each other within a second degree of freedom that is different than the first degree of freedom of movement between the first and second joint members; and
wherein the fifth and sixth joint members are configured to move relative to each other within a third degree of freedom that is different than the second degree of freedom of movement between the third and fourth joint members.

20. The orthopedic device of claim 19, wherein the first degree of freedom is translational, the second degree of freedom is rotational, and the third degree of freedom is translational.

21. The orthopedic device of claim 7. wherein the central post deforms or bends about the central axis.

22. The orthopedic device of claim 21, wherein the at least one sidewall of the opening in the first joint member is configured to limit the range of deformation of the central post.
